(19) United States
(12) Patent Application Publication
(10) Pub. No.: US 2020/0035927 A1
(43) Pub. Date: Jan. 30, 2020

(54) COMPOUND HAVING AZAINDENO[1,2-c]PHENANTHRENE RING STRUCTURE, AND ORGANIC ELECTROLUMINESCENCE DEVICE USING THE SAME

(71) Applicant: HODOGAYA CHEMICAL CO., LTD., Tokyo (JP)

(72) Inventors: Kouki KASE, Tokyo (JP); Si-In KIM, Tokyo (JP); Yuta HIRAYAMA, Tokyo (JP)

(21) Appl. No.: 16/522,107

(22) Filed: Jul. 25, 2019

(30) Foreign Application Priority Data
Jul. 25, 2018 (JP) 2018-139292

(51) Int. Cl. H01L 51/00; C07D 491/147; C09K 11/06

(52) U.S. Cl. CPC H01L 51/0071; C07D 491/147; H01L 51/5072; H01L 51/0052; H01L 51/0067; C09K 11/06

(57) ABSTRACT

A compound has an azaindeno phenanthrene ring structure, the compound being represented by the following general formula (A-1).

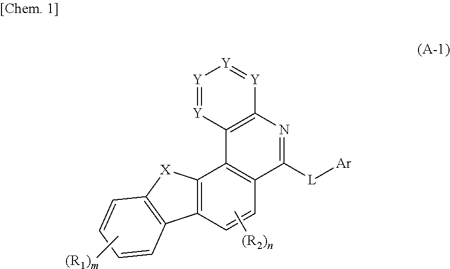

### CROSS-REFERENCE TO RELATED APPLICATION

This application claims priority to Japanese Application No. 2018-139292, filed Jul. 25, 2018, which is hereby incorporated by reference in its entirety.

### BACKGROUND

The present disclosure relates to a compound and a device suitable for an organic electroluminescent device (hereinafter, referred to as “organic EL device”), which is a self light-emitting device suitable for various display apparatuses. Specifically, the present disclosure relates to a compound having an azaindeno [1,2-c] phenanthrene ring structure and an organic EL device using the compound.

Since the organic EL device is a self light-emitting device, it is brighter than a liquid crystal device, has excellent visibility, and is capable of performing clear display, active research has been conducted on the organic EL device.

In 1987, C. W. Tang et al. (Eastman Kodak Company) have developed a device having a stacked structure in which various roles are assigned to respective materials, and put an organic EL device using an organic material to practical use. C. W. Tang et al. have formed the organic EL device by stacking a fluorescent material capable of transporting electrons and an organic material capable of transporting holes. With this structure, by injecting charges of both the electrons and holes into a fluorescent material layer to emit light, high luminance of not less than 1000 cd/m² could have been achieved with voltage of not more than 10 V (see, for example, Japanese Patent Application Laid-open No. 1996-048656 and Japanese Patent No. 3194657).

Many improvements have been made for practical use of the organic EL device until now. For example, in an electroluminescent device that subdivides roles of the stacked structure and includes an anode, a hole injection layer, a hole transport layer, a light-emitting layer, an electron transport layer, an electron injection layer, and a cathode in the stated order on a substrate, a high efficiency and durability have been achieved (see, for example, The Japan Society of Applied Physics, proceedings of the ninth workshop, pp. 55-61 (2001)).

Further, for the purpose of further improving the light emission efficiency, attempts have been made to use a triplet excitor and utilization of a phosphorescent compound is being considered (see, for example, The Japan Society of Applied Physics, proceedings of the ninth workshop, pp. 23-31 (2001)). A device using light emission by thermally activated delayed fluorescence (TADF) has also been developed. In 2011, Adachi et al. (Kyushu University) have realized the external quantum efficiency of 5.3% by a device using a thermally activated delayed fluorescence material (see, for example, Appl. Phys. Let., 98,083302 (2011)).

The light-emitting layer can also be prepared by doping a charge transport compound generally called a host material with a fluorescent compound, a phosphorescent compound, or a material emitting delayed fluorescence. Selection of an organic material in the organic EL device significantly affects various properties such as the efficiency and durability of the device (see, for example, The Japan Society of Applied Physics, proceedings of the ninth workshop, pp. 23-31 (2001)).

In the organic EL device, charges injected from both electrodes are recombined in the light-emitting layer to obtain light emission. For that reason, it is important how efficiently charges of both the holes and the electrons are transferred to the light-emitting layer. Further, by improving the electron injection property, increasing the mobility thereof, improving the hole blocking property for blocking the holes injected from the anode to increase the possibility of recombination of holes and electrons, and further confining the excitons generated in the light-emitting layer, it is possible to achieve a high light emission efficiency. Therefore, the role played by the electron transport material is important, and an electron transport material having a high electron injection property, a high mobility of electrons, a high hole blocking property, and a high durability to holes is desired.

Further, from the viewpoint of device lifetime, the heat resistance and amorphous property of the material are also important, In the case of a material having a low heat resistance, thermal decomposition occurs even at a low temperature due to heat generated at the time of driving the device, and the material is degraded. In the case of a material having a low amorphous property, crystallization of the thin film occurs even in a short time, and the device is degraded. Therefore, the material to be used is desired to have a high heat resistance and an excellent amorphous property.

A typical light-emitting material, tris (8-hydroxyquinoline) aluminum (hereinafter, referred to as “Alq₃”), is generally used as an electron transport material. However, since Alq₃ has slow electron transfer and a work function of 5.6 eV, the hole blocking property is not sufficient.

As a compound having improved properties such as an electron injection property and mobility, compounds having a benzotriazole structure have been proposed (see, for example, WO 2013/054764). However, in a device using such a compound as an electron injection layer or an electron transport layer, although the light emission efficiency and the like have been improved, which are still not sufficient, further reduction in the drive voltage and further improvement of the light emission efficiency are desired.

Further, as an electron transport material excellent in hole blocking property, 3-(4-biphenylyl)-4-phenyl-5-(4-t-butylphenyl)-1,2,4-triazole (hereinafter, referred to as “TAZ”) has been proposed (see, for example, Patent Registration No. 2734341).

TAZ has a large work function of 6.6 eV and a high hole blocking property. For this reason, TAZ is used as an electron-transporting a hole blocking layer to be stacked on the side of a cathode of a fluorescent light-emitting layer or a phosphorescent light-emitting layer prepared by vacuum deposition or coating, and contributes to the high efficiency of the organic EL device (see, for example, proceedings of the 50th Meeting of The Japan Society of Applied Physics and Related Societies 28p-A-6, p. 1413 (2003)).

However, the low electron transportability is a major issue in TAZ, and it has been necessary to prepare an organic EL device by combining TAZ with an electron transport material having a more high electron transportability (see, for example, Molecular electronics and bioelectronics, Vol. 11, No. 1, pp. 13-19 (2000)).

Further, also bathocuproine (hereinafter, referred to as “BCP”) has a large work function of 6.7 eV and a high hole blocking property. However, since BCP has a low glass transition point (Tg) of 83° C., the thin film lacks stability. Therefore, BCP does not sufficiently function as a hole blocking layer.

Any of the above-mentioned materials has poor film stability or insufficient hole blocking function. In order to improve the device characteristics of an organic EL device, an organic compound that is excellent in electron injection/transporting property and hole blocking property and has high stability in a thin film state is desired.

### SUMMARY

In view of the circumstances as described above, it is desired to provide, as a material for organic EL device having a high efficiency and high durability, an organic compound having excellent characteristics, i.e., an organic compound that is excellent in electron injection/transporting property and hole blocking property and has high stability in a thin film state, and provide an organic EL device having a high efficiency and high durability by using this compound.

Examples of physical characteristics that an organic material to be provided by an embodiment of the present disclosure should have include (1) favorable electron injecting property, (2) high electron mobility, (3) excellent hole blocking property, (4) stable thin film state, and (5) excellent heat resistance. Further, examples of physical characteristics that an organic EL device to be provided by an embodiment of the present disclosure should have include (1) a high light emission efficiency and high power efficiency, (2) a low light-emission initiation voltage, (3) a low practical drive voltage, and (4) being long lifetime.

In order to achieve the above-mentioned object, the present inventors have designed and chemically synthesized a compound having azaindeno [1,2-c] phenanthrene ring structure, focusing on the fact that a nitrogen atom of an azaindeno [1,2-c] phenanthrene ring structure having an electron affinity has the ability to coordinate to a metal and is excellent in heat resistance, experimentally produced various organic EL devices using the compound, and conducted intensive evaluation of the characteristics of the device, thereby completing a compound and an organic EL device according to an embodiment of the present disclosure.

1) That is, according to an embodiment of the present disclosure, there is provided a compound having an azaindeno [1,2-c] phenanthrene ring structure represented by the following general formula (A-1).

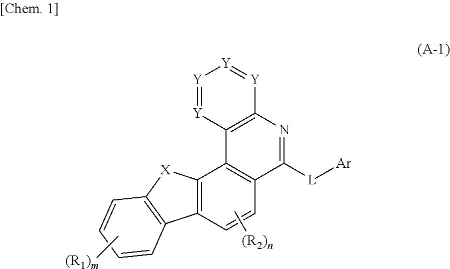

(wherein, Ys may be the same as or different from each other, and each represent a hydrogen atom, a deuterium atom, a fluorine atom, a chlorine atom, a cyano group, a nitro group, a trimethylsilyl group, a carbon atom having a triphenylsilyl group, a carbon atom having a substituted or unsubstituted aromatic hydrocarbon group, a carbon atom having a substituted or unsubstituted aromatic heterocyclic group, a carbon atom having a substituted or unsubstituted fused polycyclic aromatic group, a carbon atom having a linear or branched alkyl group having 1 to 6 carbon atoms which may have a substituent, a carbon atom having a cycloalkyl group having 5 to 10 carbon atoms which may have a substituent, a carbon atom having a linear or branched alkenyl group having 2 to 6 carbon atoms which may have a substituent, a carbon atom having a linear or branched alkyloxy group having 1 to 6 carbon atoms which may have a substituent, a carbon atom having a cycloalkyloxy group having 5 to 10 carbon atoms which may have a substituent, or a nitrogen atom,

L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁ and R₂ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,

X represents an oxygen atom or a sulfur atom,

m represents an integer of 0 to 4, and

n represents an integer of 0 to 2.

However, where m or n is an integer of two or more, at least one of Ys may be a nitrogen atom, and a plurality of R₁ or a plurality of R₂, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

2) Further, according to an embodiment of the present disclosure, there is provided the compound having an azaindeno [1,2-c] phenanthrene ring structure according to 1) above, which is represented by the following general formula (A-2).

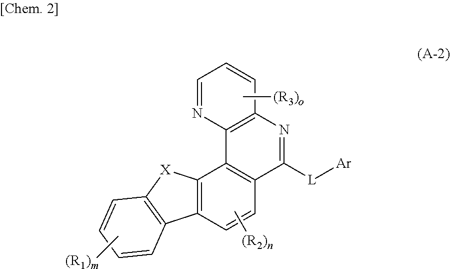

(wherein, L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁, R₂, and R₃ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,

X represents an oxygen atom or a sulfur atom,

m represents an integer of 0 to 4,

n represents an integer of 0 to 2, and

o represents an integer of 0 to 3.

However, where m, n, or o is an integer of two or more, a plurality of R₁, a plurality of R₂, or a plurality of R₃, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

3) Further, according to an embodiment of the present disclosure, there is provided the compound having an azaindeno [1,2-c] phenanthrene ring structure according to 1) above, which is represented by the following general formula (A-3).

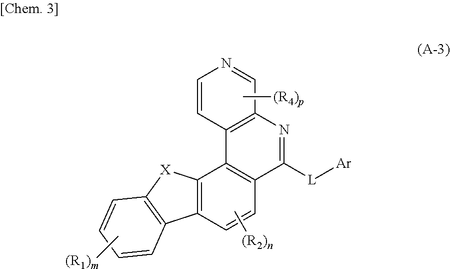

(wherein, L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁, R₂, and R₄ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,

X represents an oxygen atom or a sulfur atom,

m represents an integer of 0 to 4,

n represents an integer of 0 to 2, and

p represents an integer of 0 to 3.

However, where m, n, or p is an integer of two or more, a plurality of R₁, a plurality of R₂, or a plurality of R₄, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

4) Further, according to an embodiment of the present disclosure, there is provided the compound having an azaindeno [1,2-c] phenanthrene ring structure according to 1) above, which is represented by the following general formula (A-4).

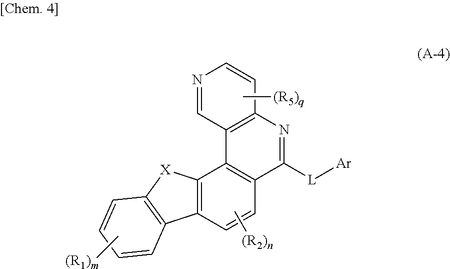

(wherein, L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁, R₂, and R₅ may be the same as or different from each other, and each represents a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,

X represents an oxygen atom or a sulfur atom,

m represents an integer of 0 to 4,

n represents an integer of 0 to 2, and

q represents an integer of 0 to 3.

However, where m, n, or q is an integer of 2 or more, a plurality of R₁, a plurality of R₂, or a plurality of R₅, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

5) Further, according to an embodiment of the present disclosure, there is provided the compound having an azaindeno [1,2-c] phenanthrene ring structure according to 1) above, which is represented by the following general formula (A-5).

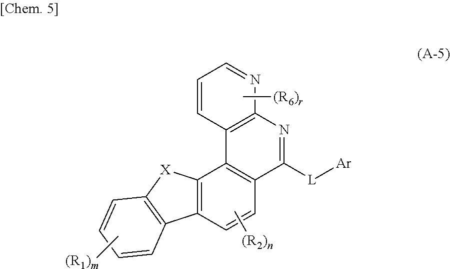

(wherein, L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁, R₂, and R₆ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,

X represents an oxygen atom or a sulfur atom,

m represents an integer of 0 to 4,

n represents an integer of 0 to 2, and

r represents an integer of 0 to 3.

However, m, n, or r is an integer of two or more, a plurality of R₁, a plurality of R₂, or a plurality of R₆, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

6) Further, according to an embodiment of the present disclosure, there is provided the compound having an azaindeno [1,2-c] phenanthrene ring structure according to 1) above, which is represented by the following general formula (A-6).

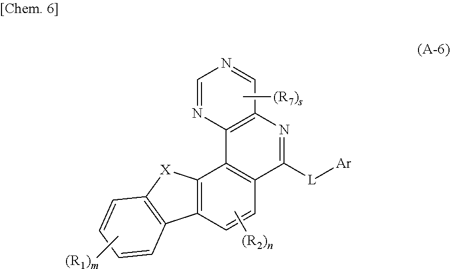

(wherein, L represents a divalent group of a substituted or unsubstituted aromatic hydrocarbon group, a substituted or unsubstituted aromatic heterocyclic group, or a substituted or unsubstituted fused polycyclic aromatic group,

Ar represents a hydrogen atom, a substituted or unsubstituted aromatic hydrocarbon group, a substituted or unsubstituted aromatic heterocyclic group, or a substituted or unsubstituted fused polycyclic aromatic group,

R₁, R₂, and R₇ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a sub stituent,

X represents an oxygen atom or a sulfur atom,

m represents an integer of 0 to 4,

n represents an integer of 0 to 2, and

s represents an integer of 0 to 2.

However, where m, n, or s is an integer of two or more, a plurality of R₁, a plurality of R₂, or a plurality of R₇, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

7) Further, according to an embodiment of the present disclosure, there is provided the compound having an azaindeno [1,2-c] phenanthrene ring structure according to any one of 2) to 6) above, in which L is represented by the following structural formula (B-1), (B-2), or (B-3), and two portions among R₁₁ to R₁₅, among R₁₆ to R₂₁, or among R₂₂ to R₂₉ are binding sites.

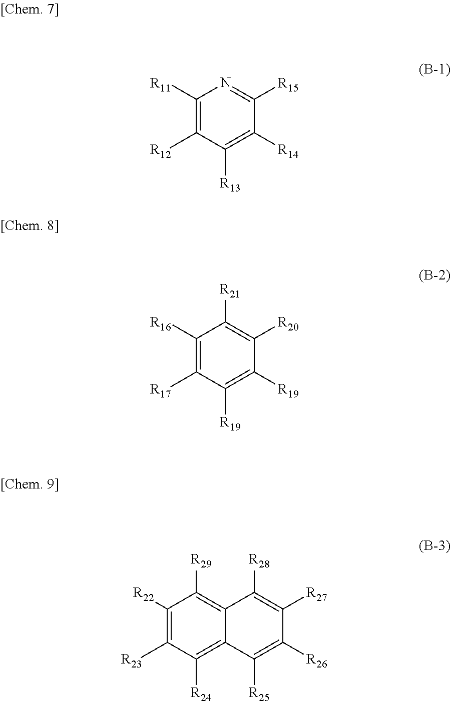

(wherein, R₁₁ to R₂₉ may be the same as or different from each other, and each represent a linking group as a binding site, a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent.)

8) Further, according to an embodiment of the present disclosure, there is provided an organic electroluminescent device, including:

a pair of electrodes; and

at least one organic layer sandwiched between the pair of electrodes,

the compound having an azaindeno phenanthrene ring structure according to any one of 1) to 7) above being used as a constituent material of the at least one organic layer.

9) Further, according to an embodiment of the present disclosure, there is provided the organic electroluminescent device according to 8) above, in which

the organic layer containing the compound having an azaindeno phenanthrene ring structure as the constituent material is an electron transport layer.

10) Further, according to an embodiment of the present disclosure, there is provided the organic electroluminescent device according to 8) above, in which

the organic layer containing the compound having an azaindeno phenanthrene ring structure as the constituent material is a hole blocking layer.

11) Further, according to an embodiment of the present disclosure, there is provided the organic electroluminescent device according to 8) above, in which

the organic layer containing the compound having an azaindeno phenanthrene ring structure as the constituent material is a light-emitting layer.

12) Further, according to an embodiment of the present disclosure, there is provided the organic electroluminescent device according to 8) above, in which

the organic layer containing the compound having an azaindeno phenanthrene ring structure as the constituent material is an electron injection layer.

Ys in the general formula (A-1) may be the same as or different from each other, and each represent a hydrogen atom, a deuterium atom, a fluorine atom, a chlorine atom, a carbon atom having a cyano group, a nitro group, a trimethylsilyl group, or a triphenylsilyl group, a carbon atom having a substituted or unsubstituted aromatic hydrocarbon group, a carbon atom having a substituted or unsubstituted aromatic heterocyclic group, a carbon atom having a substituted or unsubstituted fused polycyclic aromatic group, a carbon atom having a linear or branched alkyl group having 1 to 6 carbon atoms which may have a substituent, a carbon atom having a cycloalkyl group having 5 to 10 carbon atoms which may have a substituent, a carbon atom having a linear or branched alkenyl group having 2 to 6 carbon atoms which may have a substituent, a carbon atom having a linear or branched alkyloxy group having 1 to 6 carbon atoms which may have a substituent, a carbon atom having a cycloalkyloxy group having 5 to 10 carbon atoms which may have a substituent, or a nitrogen atom.

The “aromatic hydrocarbon group”, “aromatic heterocyclic group”, or “fused polycyclic aromatic group” in the “carbon atom having a substituted or unsubstituted aromatic hydrocarbon group”, “carbon atom having a substituted or unsubstituted aromatic heterocyclic group”, or “carbon atom having a substituted or unsubstituted fused polycyclic aromatic group” represented by Y in the general formula (A-1) can be selected from, specifically, the group consisting of an aryl group having 6 to 30 carbon atoms and a heteroaryl group having 2 to 30 carbon atoms in addition to a phenyl group, a biphenylyl group, a terphenylyl group, a naphthyl group, an anthracenyl group, a phenanthrenyl group, a fluorenyl group, a spirobifluorenyl group, an indenyl group, a pyrenyl group, a perylenyl group, a fluoranthenyl group, a triphenylenyl group, a pyridyl group, a pyrimidinyl group, a triazinyl group, a furyl group, a pyrrolyl group, a thienyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzimidazolyl group, a pyrazolyl group, a dibenzofuranyl group, a dibenzothienyl group, a naphthyridinyl group, a phenanthrolinyl group, an acridinyl group, and a carbolinyl group, and benzene rings substituted with these substituents or a plurality of substituents substituted on the same benzene ring may be bonded to each other via a single bond, a substituted or unsubstituted methylene group, a substituted or unsubstituted amino group, an oxygen atom, or a sulfur atom to form a ring.

Specific examples of the “carbon atom having a linear or branched alkyl group having 1 to 6 carbon atoms”, “carbon atom having a cycloalkyl group having 5 to 10 carbon atoms”, or “carbon atom having a linear or branched alkenyl group having 2 to 6 carbon atoms” in the “carbon atom having a linear or branched alkyl group having 1 to 6 carbon atoms which may have a substituent”, “carbon atom having a cycloalkyl group having 5 to 10 carbon atoms which may have a substituent”, “carbon atom having a linear or branched alkenyl group having 2 to 6 carbon atoms which may have a substituent” represented by Y in the general formula (A-1) include a methyl group, an ethyl group, an n-propyl group, an isopropyl group, an n-butyl group, an isobutyl group, a tert-butyl group, an n-pentyl group, an isopentyl group, a neopentyl group, an n-hexyl group, a cyclopentyl group, a cyclohexyl group, a 1-adamantyl group, a 2-adamantyl group, a vinyl group, an allyl group, an isopropenyl group, and a 2-butenyl group, and benzene rings substituted with these substituents or a plurality of substituents substituted on the same benzene ring may be bonded to each other via a single bond, a substituted or unsubstituted methylene group, a substituted or unsubstituted amino group, an oxygen atom, or a sulfur atom to form a ring.

Specific examples of the “carbon atom having a linear or branched alkyloxy group having 1 to 6 carbon atoms” or “carbon atom having a cycloalkyloxy group having 5 to 10 carbon atoms” in the “carbon atom having a linear or branched alkyloxy group having 1 to 6 carbon atoms which may have a substituent” or “carbon atom having a cycloalkyloxy group having 5 to 10 carbon atoms which may have a substituent” represented by Y in the general formula (A-1) include a methyloxy group, an ethyloxy group, an n-propyloxy group, an isopropyloxy group, an n-butyloxy group, a tert-butyloxy group, an n-pentyloxy group, an n-hexyloxy group, a cyclopentyloxy group, a cyclohexyloxy group, a cycloheptyloxy group, a cyclooctyloxy group, a 1-adamantyloxy group, and a 2-adamantyloxy group, and benzene rings substituted with these substituents or a plurality of substituents substituted on the same benzene ring may be bonded to each other via a single bond, a substituted or unsubstituted methylene group, a substituted or unsubstituted amino group, an oxygen atom, or a sulfur atom to form a ring.

Specific examples of the “sub stituent” in the “carbon atom having a substituted aromatic hydrocarbon group”, “carbon atom having a substituted aromatic heterocyclic group”, “carbon atom having a substituted fused polycyclic aromatic group”, “carbon atom having a linear or branched alkyl group having 1 to 6 carbon atoms which may have a substituent”, “carbon atom having a cycloalkyl group having 5 to 10 carbon atoms which may have a substituent”, “carbon atom having a linear or branched alkenyl group having 2 to 6 carbon atoms which may have a substituent”, “carbon atom having a linear or branched alkyloxy group having 1 to 6 carbon atoms which may have a substituent”, or “carbon atom having a cycloalkyloxy group having 5 to 10 carbon atoms which may have a substituent” represented by Y in the general formula (A-1) include a deuterium atom, a cyano group, a nitro group; a halogen atom such as a fluorine atom, a chlorine atom, a bromine atom, and an iodine atom; a silyl group such as a trimethylsilyl group and a triphenylsilyl group; a linear or branched alkyl group having 1 to 6 carbon atoms such as a methyl group, an ethyl group, and a propyl group; a linear or branched alkyloxy group having 1 to 6 carbon atoms such as a methyloxy group, an ethyloxy group, and a propyloxy group; an alkenyl group such as a vinyl group and an allyl group; an aryloxy group such as a phenyloxy group and a tolyloxy group; an arylalkyloxy group such as a benzyloxy group and a phenethyloxy group; aromatic hydrocarbon group or fused polycyclic aromatic group such as a phenyl group, a biphenylyl group, a terphenylyl group, a naphthyl group, an anthracenyl group, a phenanthrenyl group, a fluorenyl group, a spirobifluorenyl group, an indenyl group, a pyrenyl group, a perylenyl group, a fluoranthenyl group, and a triphenylenyl group; and an aromatic heterocyclic group such as a pyridyl group, a thienyl group, a furyl group, a pyrrolyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzimidazolyl group, a pyrazolyl group, a dibenzofuranyl group, a dibenzothienyl group, and a carbolinyl group. These substituents may be further substituted by the substituents exemplified above. Further, benzene rings substituted with these substituents or a plurality of substituents substituted on the same benzene ring may be bonded to each other via a single bond, a substituted or unsubstituted methylene group, a substituted or unsubstituted amino group, an oxygen atom, or a sulfur atom to form a ring.

Ar in the general formulae (A-1) to (A-6) represents a hydrogen atom, a substituted or unsubstituted aromatic hydrocarbon group, a substituted or unsubstituted aromatic heterocyclic group, or a substituted or unsubstituted fused polycyclic aromatic group.

Examples of the “aromatic hydrocarbon group”, “aromatic heterocyclic group”, or “fused polycyclic aromatic group” in the “substituted or unsubstituted aromatic hydrocarbon group”, “substituted or unsubstituted aromatic heterocyclic group”, or “substituted or unsubstituted fused polycyclic aromatic group” represented by Ar in the general formulae (A-1) to (A-6) include the same ones as described for the “aromatic hydrocarbon group”, “aromatic heterocyclic group”, or “fused polycyclic aromatic group” in Y in the general formula (A-1), and aspects similar to those of the “aromatic hydrocarbon group”, “aromatic heterocyclic group”, or “fused polycyclic aromatic group” in Y in the general formula (A-1) can be taken.

Examples of the “substituent” in the “substituted aromatic hydrocarbon group”, “substituted aromatic heterocyclic group”, or “substituted fused polycyclic aromatic group” represented by Ar in the general formulae (A-1) to (A-6) include the same ones as described for the “substituent” in Y in the general formula (A-1), and aspects similar to those of the “substituent” in Y in the general formula (A-1) can be taken.

L in the general formulae (A-1) to (A-6) represents a divalent group of a substituted or unsubstituted aromatic hydrocarbon group, a substituted or unsubstituted aromatic heterocyclic group, or a substituted or unsubstituted fused polycyclic aromatic group.

Examples of the “aromatic hydrocarbon group”, “aromatic heterocyclic group”, or “fused polycyclic aromatic group” in the “substituted or unsubstituted aromatic hydrocarbon group”, “substituted or unsubstituted aromatic heterocyclic group”, or “substituted or unsubstituted fused polycyclic aromatic group” represented by L in the general formulae (A-1) to (A-6) include the same ones as described for the “aromatic hydrocarbon group”, “aromatic heterocyclic group”, or “fused polycyclic aromatic group” in Y in the general formula (A-1), and aspects similar to those of the “aromatic hydrocarbon group”, “aromatic heterocyclic group”, or “fused polycyclic aromatic group” in Y in the general formula (A-1) can be taken.

Examples of the “substituent” in the “substituted aromatic hydrocarbon group”, “substituted aromatic heterocyclic group”, or “substituted fused polycyclic aromatic group” represented by L in the general formulae (A-1) to (A-6) include the same ones as described for the “substituent” in Y in the general formula (A-1), and aspects similar to those of the “substituent” in Y in the general formula (A-1) can be taken.

R₁ to R₇ in the general formulae (A-1) to (A-6) and R₁₁ to R₂₉ in the structural formulae (B-1), (B-2), and (B-3) may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent.

Examples of the “aromatic hydrocarbon group”, “aromatic heterocyclic group”, or “fused polycyclic aromatic group” in the “substituted or unsubstituted aromatic hydrocarbon group”, “substituted or unsubstituted aromatic heterocyclic group”, or “substituted or unsubstituted fused polycyclic aromatic group” represented by R₁ to R₇ in the general formulae (A-1) to (A-6) and R₁₁ to R₂₉ in the structural formulae (B-1), (B-2), and (B-3) include the same ones as described for the “aromatic hydrocarbon group”, “aromatic heterocyclic group”, or “fused polycyclic aromatic group” in Y in the general formula (A-1), and aspects similar to those of the “aromatic hydrocarbon group”, “aromatic heterocyclic group”, or “fused polycyclic aromatic group” in Y in the general formula (A-1) can be taken.

Examples of the “linear or branched alkyl group having 1 to 6 carbon atoms”, “cycloalkyl group having 5 to 10 carbon atoms”, or “linear or branched alkenyl group having 2 to 6 carbon atoms” in the “a linear or branched alkyl group having 1 to 6 carbon atoms which may have a substituent”, “a cycloalkyl group having 5 to 10 carbon atoms which may have a substituent”, or “a linear or branched alkenyl group having 2 to 6 carbon atoms which may have a substituent” represented by R₁ to R₇ in the general formulae (A-1) to (A-6) and R₁₁ to R₂₉ in the structural formulae (B-1), (B-2), and (B-3) include the same ones as described for the “linear or branched alkyl group having 1 to 6 carbon atoms”, “cycloalkyl group having 5 to 10 carbon atoms”, or “linear or branched alkenyl group having 2 to 6 carbon atoms” in Y in the general formula (A-1), and aspects similar to those of the “linear or branched alkyl group having 1 to 6 carbon atoms”, “cycloalkyl group having 5 to 10 carbon atoms”, or “linear or branched alkenyl group having 2 to 6 carbon atoms” in Y in the general formula (A-1) can be taken.

Examples of the “linear or branched alkyloxy group having 1 to 6 carbon atoms” or “cycloalkyloxy group having 5 to 10 carbon atoms” in the “linear or branched alkyloxy group having 1 to 6 carbon atoms which may have a substituent” or “cycloalkyloxy group having 5 to 10 carbon atoms which may have a substituent” represented by R₁ to R₇ in the general formulae (A-1) to (A-6) and R₁₁ to R₂₉ in the structural formulae (B-1), (B-2), and (B-3) include the same ones as described for the “linear or branched alkyloxy group having 1 to 6 carbon atoms” or “cycloalkyloxy group having 5 to 10 carbon atoms” in Y in the general formula (A-1), and aspects similar to those of the “linear or branched alkyloxy group having 1 to 6 carbon atoms” or “cycloalkyloxy group having 5 to 10 carbon atoms” in Y in the general formula (A-1) can be taken.

Examples of the “substituent” in the “substituted aromatic hydrocarbon group”, “substituted aromatic heterocyclic group”, “substituted fused polycyclic aromatic group”, “linear or branched alkyl group having 1 to 6 carbon atoms which may have a substituent”, “cycloalkyl group having 5 to 10 carbon atoms which may have a substituent”, “linear or branched alkenyl group having 2 to 6 carbon atoms which may have a substituent”, “linear or branched alkyloxy group having 1 to 6 carbon atoms which may have a substituent”, or “cycloalkyloxy group having 5 to 10 carbon atoms which may have a substituent” represented by R₁ to R₇ in the general formulae (A-1) to (A-6) and R₁₁ to R₂₉ in the structural formulae (B-1), (B-2), and (B-3) include the same ones as described for the “substituent” in Y in the general formula (A-1), and aspects similar to those of the “substituent” in Y in the general formula (A-1) can be taken.

A compound having an azaindeno [1,2-c] phenanthrene ring structure represented by the general formula (A-1), which is suitably used for an organic EL device according to an embodiment of the present disclosure, can be used as a constituent material of an electron injection layer, an electron transport layer, or a hole blocking layer of the organic EL device. Such a compound has high electron mobility and is favorable as a material of an electron injection layer or an electron transport layer.

Since a material for organic EL device excellent in electron injection/transporting property, thin film stability, and durability is used for the organic EL device according to the embodiment of the present disclosure, the electron transport efficiency from an electron transport layer to a light-emitting layer is improved, the light emission efficiency is improved, and the drive voltage is reduced as compared with the existing organic EL device. Thus, it has become possible to improve the durability of the organic EL device, and realize an organic EL device having a high efficiency, a low drive voltage, a long lifetime.

### ADVANTAGEOUS EFFECTS OF INVENTION

The organic EL device according to the embodiment of the present disclosure is capable of efficiently injecting/transporting electrons from an electron transport layer to a light-emitting layer because a compound having a specific azaindeno [1,2-c] phenanthrene ring structure that can effectively express the role of electron injection/transport has been selected. Thus, it is possible to realize an organic EL device that is excellent in electron injection/transporting property, thin film stability, and durability and has a high efficiency, a low drive voltage, and a long lifetime. In accordance with the present disclosure, it is possible to improve the light emissi on efficiency, drive voltage, and durability of the existing organic EL device.

These and other objects, features and advantages of the present disclosure will become more apparent in light of the following detailed description of best mode embodiments thereof, as illustrated in the accompanying drawings.

### BRIEF DESCRIPTION OF DRAWINGS

FIG. 1 is a diagram showing a configuration of organic EL devices according to Examples 5 and 6 and Comparative Examples 1 to 3.

### DETAILED DESCRIPTION OF EMBODIMENTS

A compound having an azaindeno [1,2-c] phenanthrene ring structure according to an embodiment of the present disclosure is a novel compound. The compound can be synthesized in accordance with a method known per se as follows, for example (see, for example, Korean patent publication No. 2010/090139).

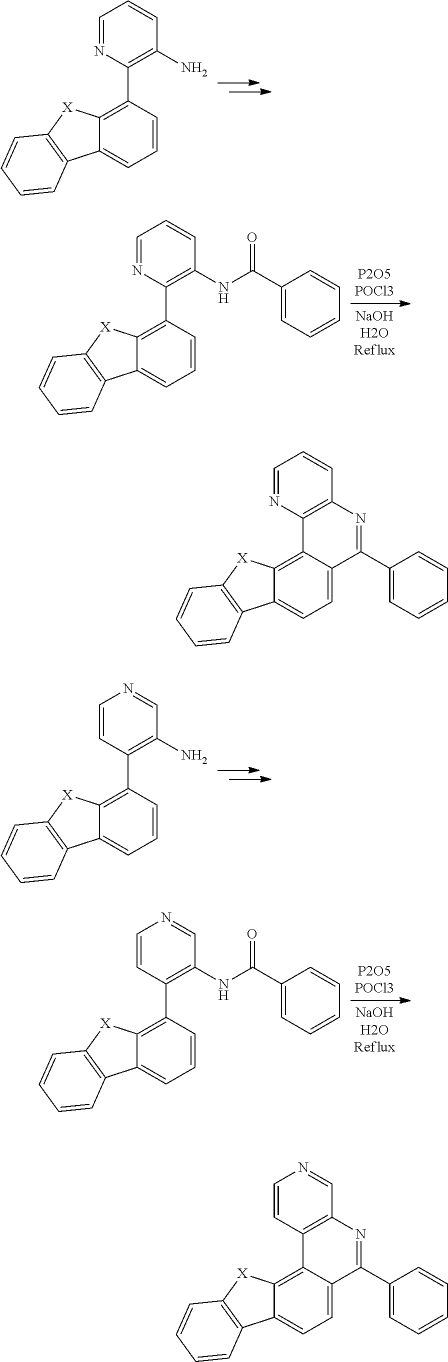

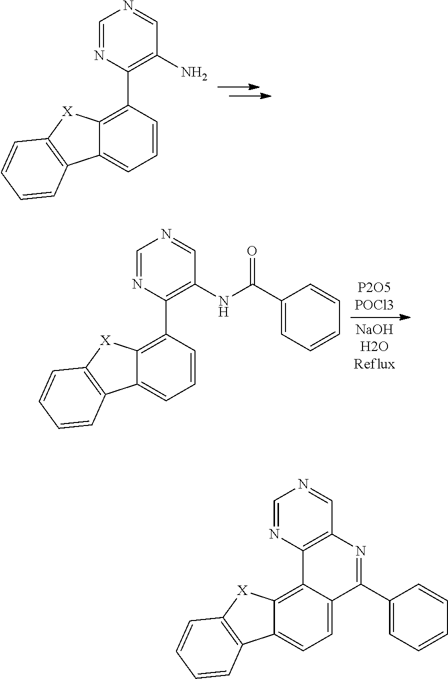

Specific examples of favorable compounds among compounds having the azaindeno [1,2-c] phenanthrene ring structure represented by the general formula(A-1), which is suitably used for the organic EL device according to the embodiment of the present disclosure, are shown below. However, the present disclosure is not limited thereto.

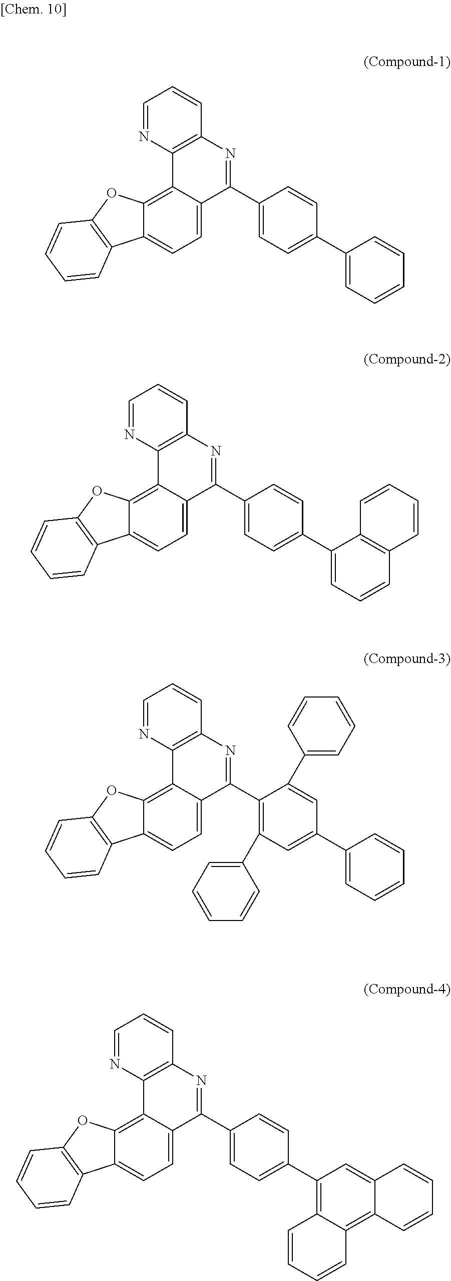

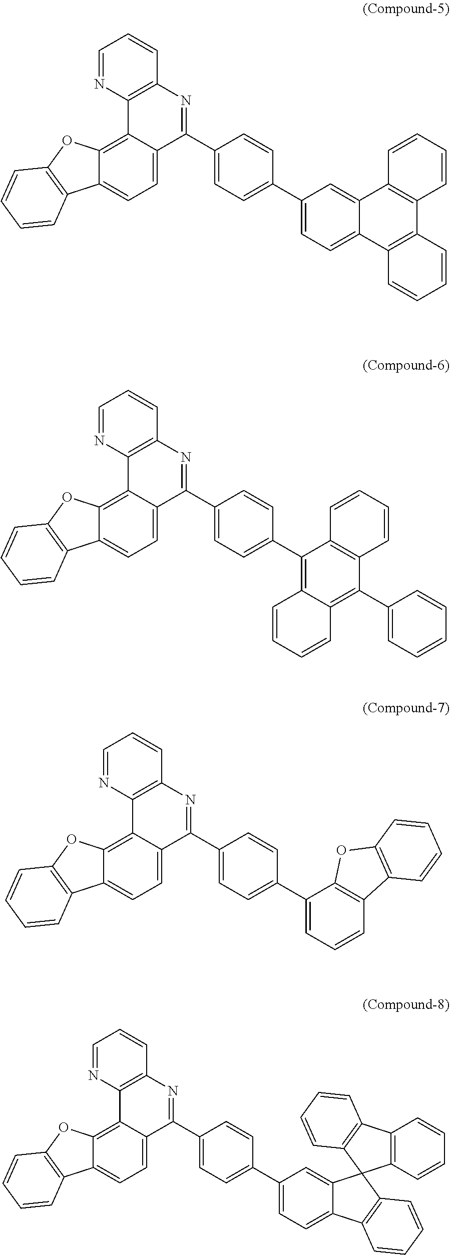

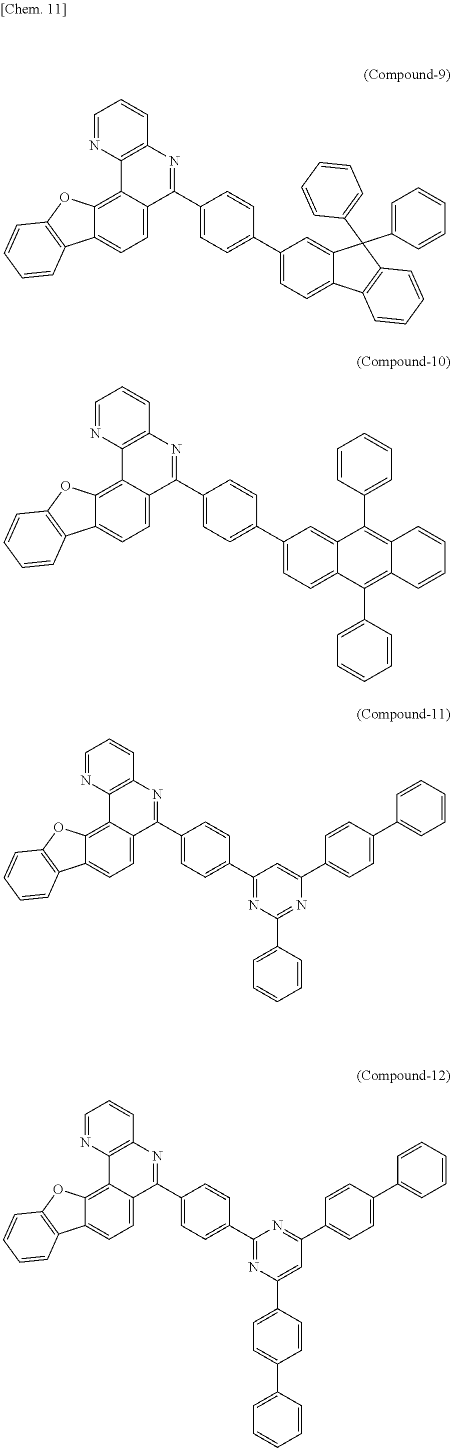

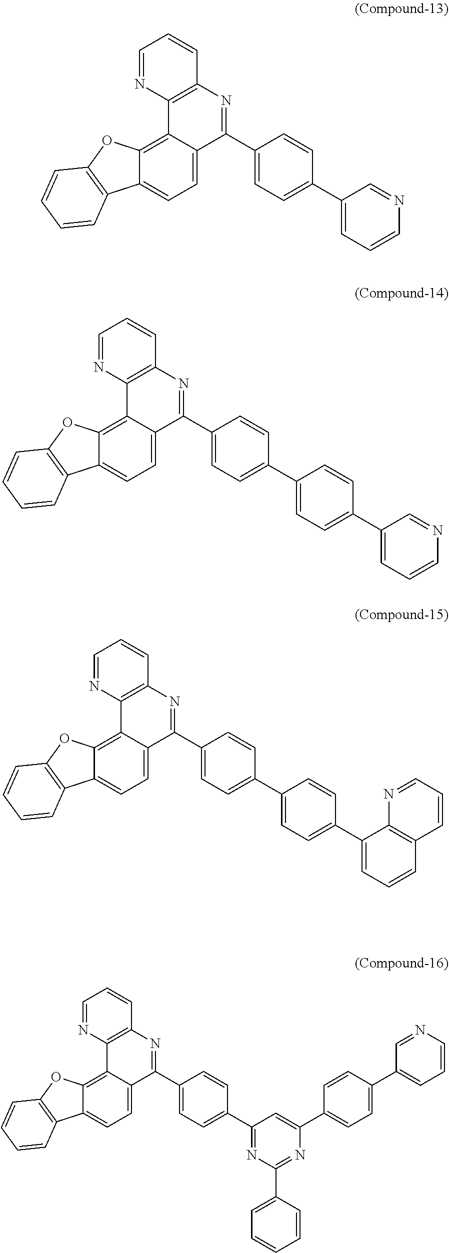

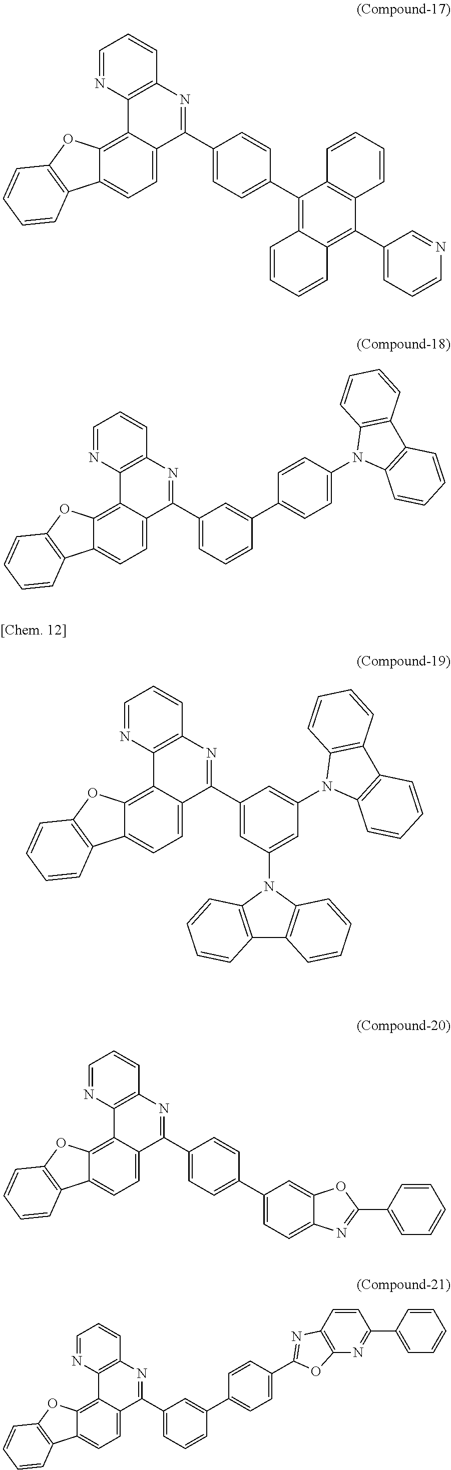

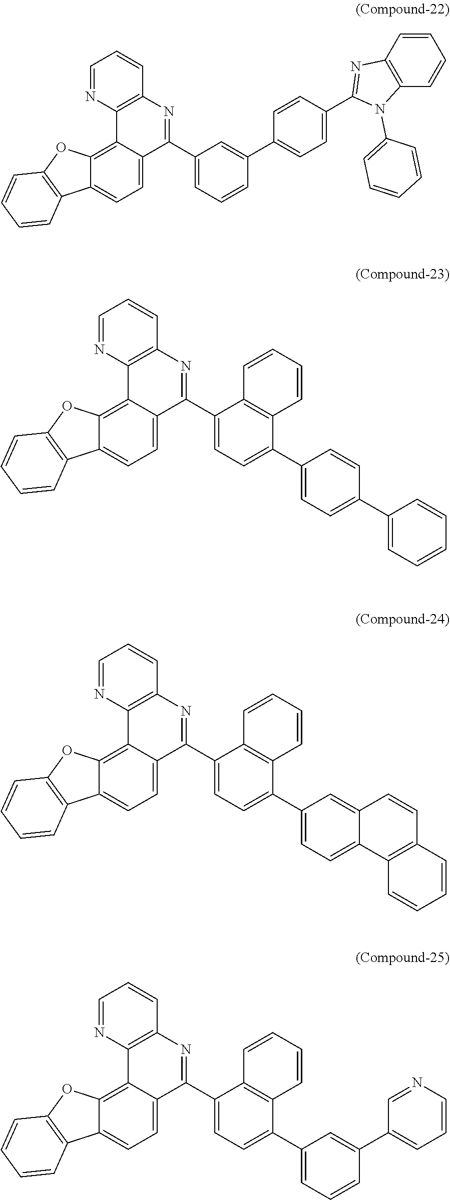

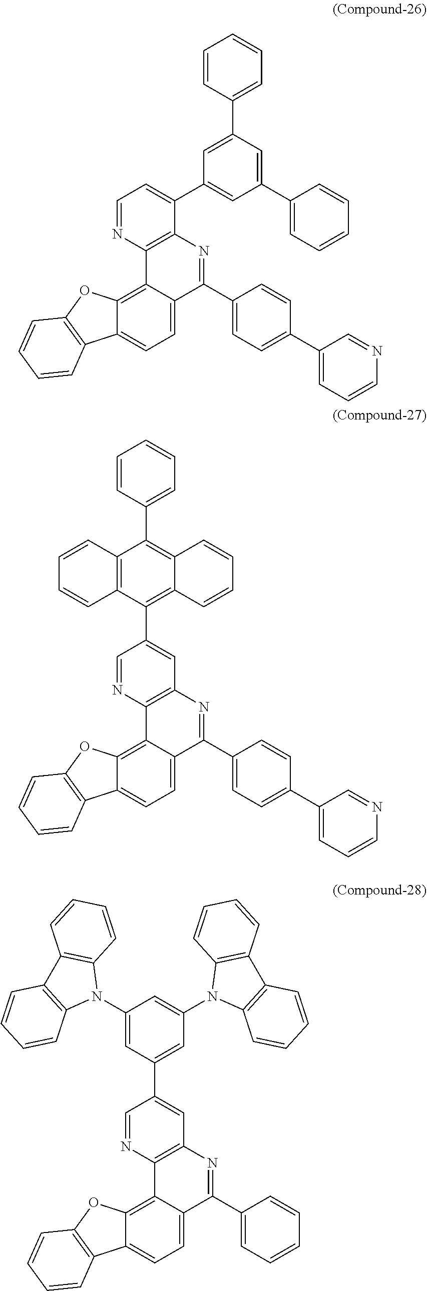

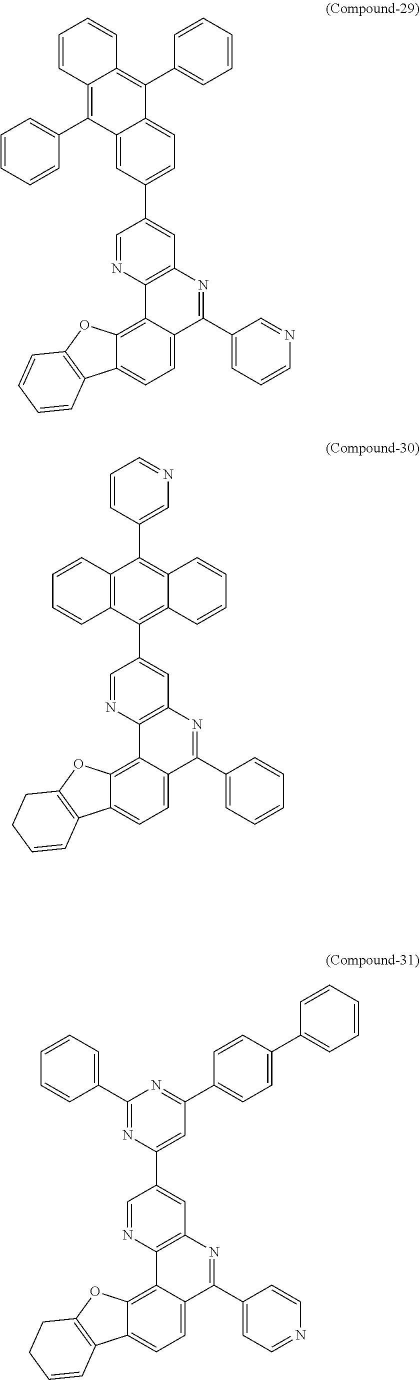

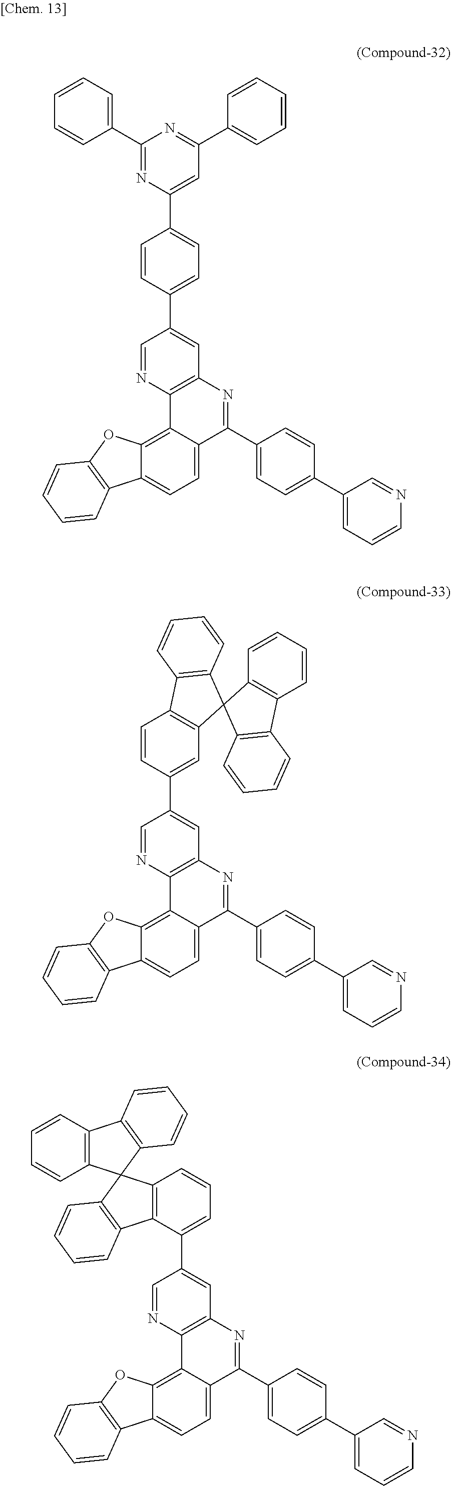

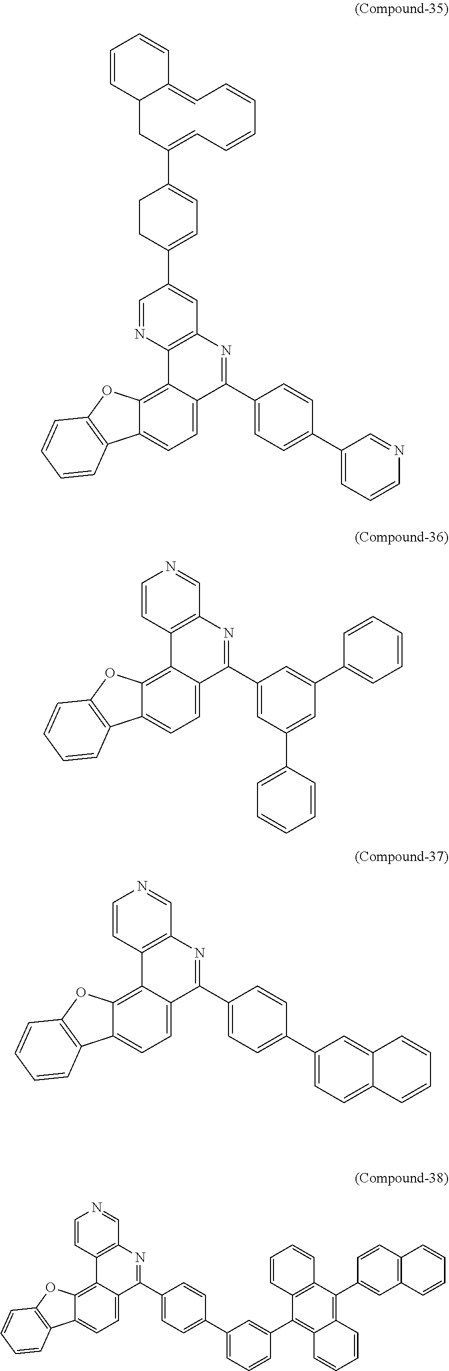

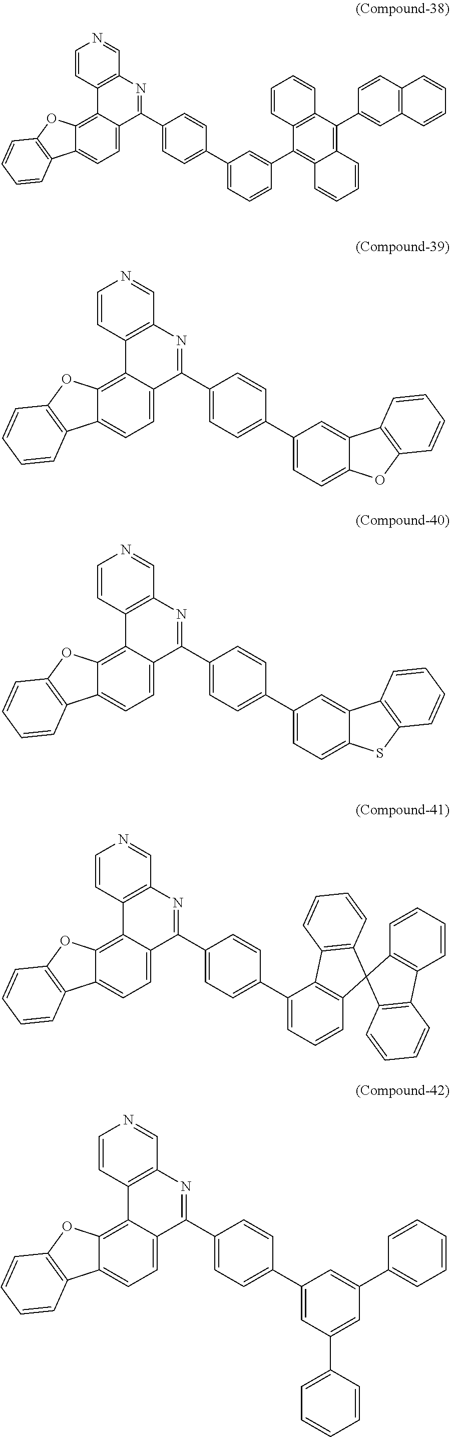

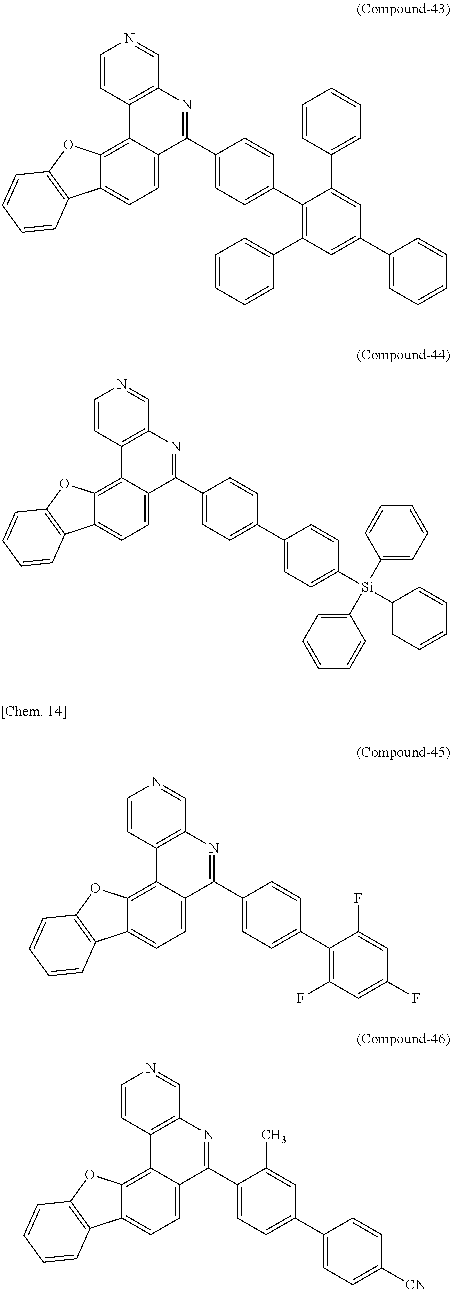

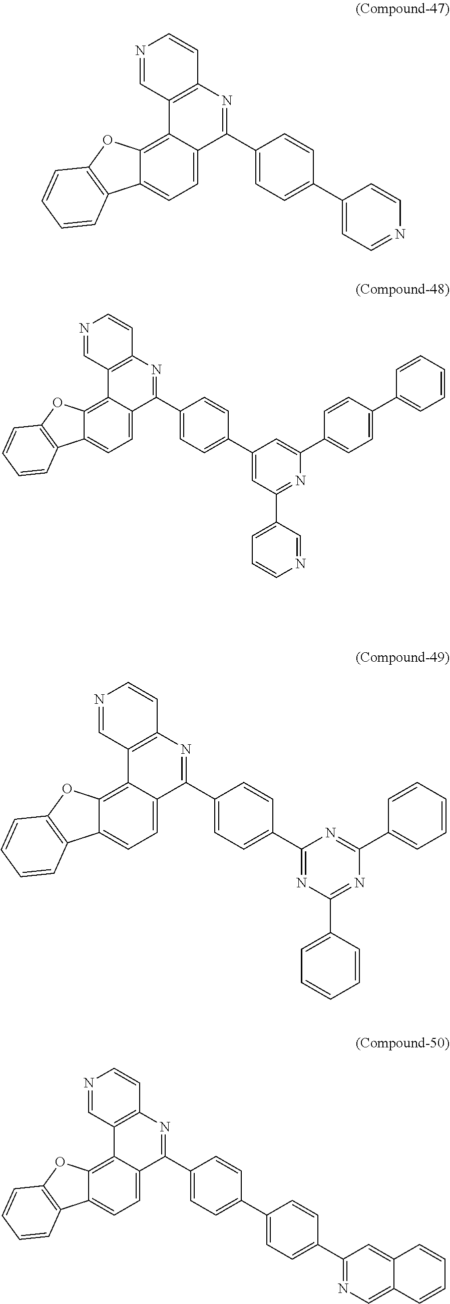

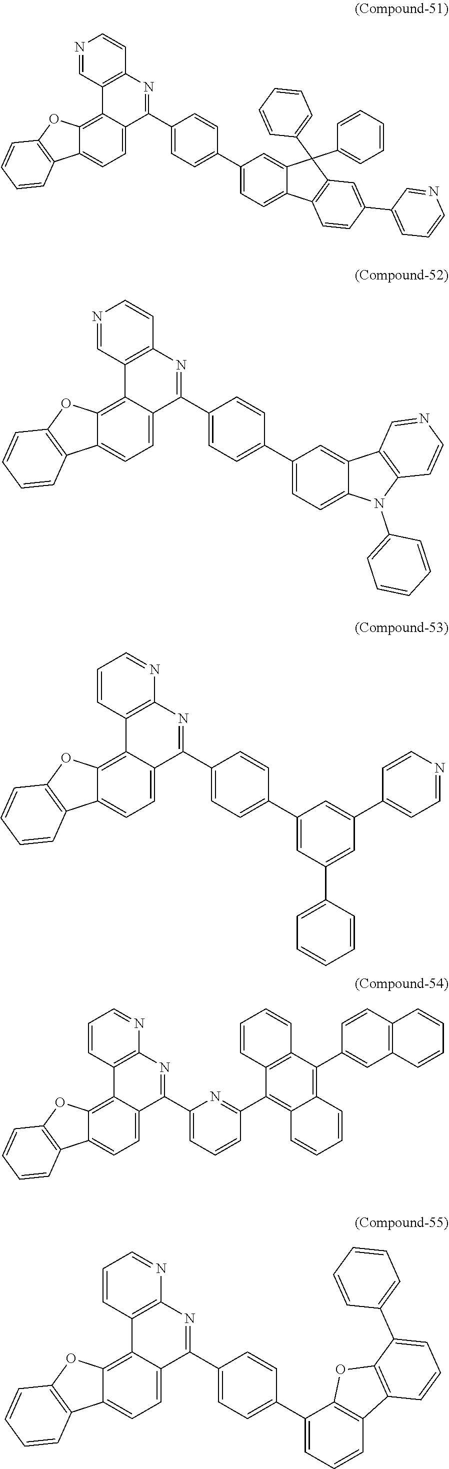

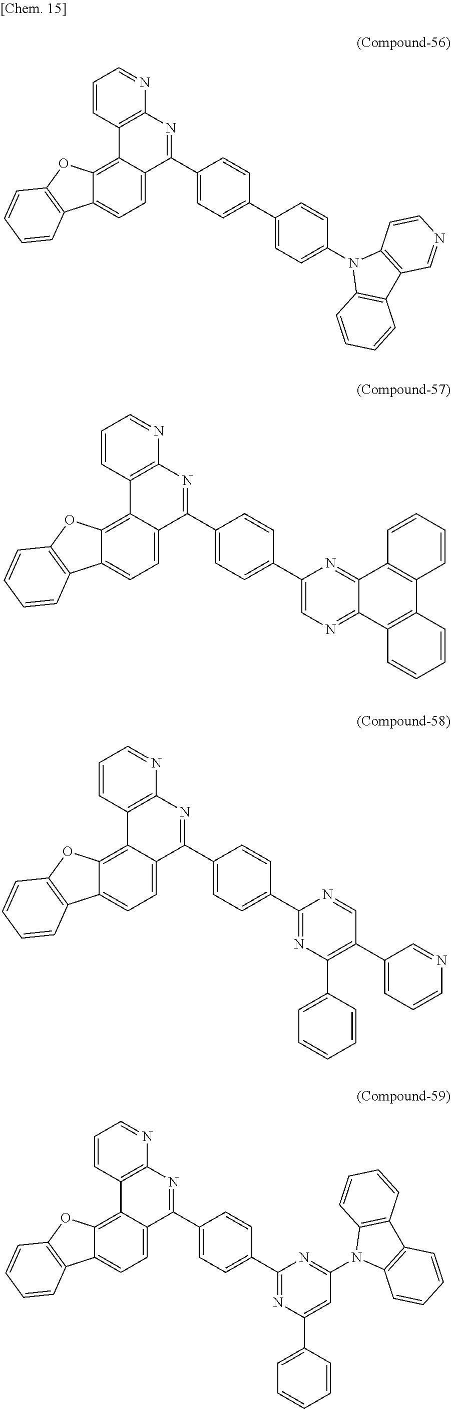

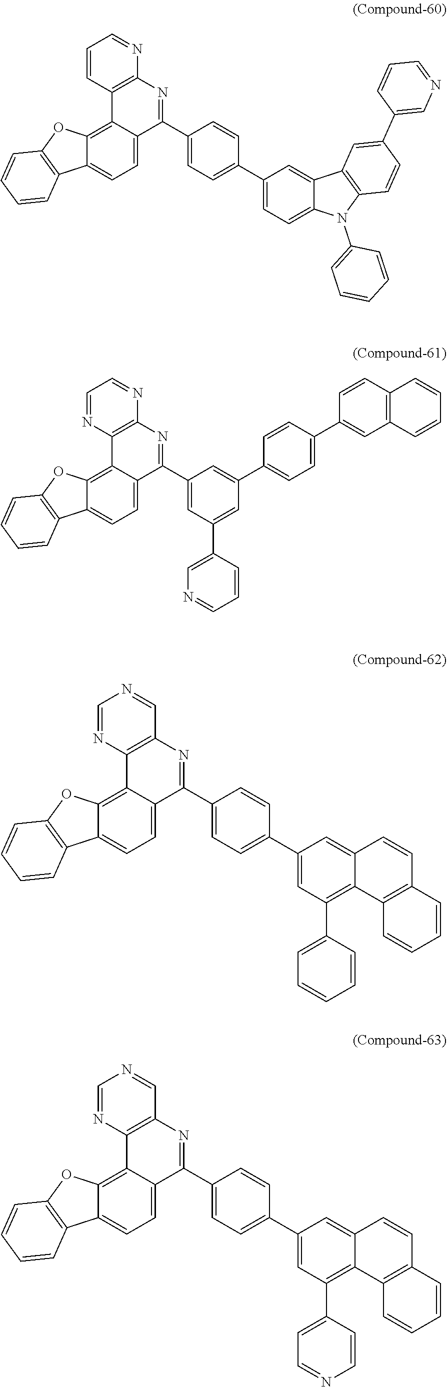

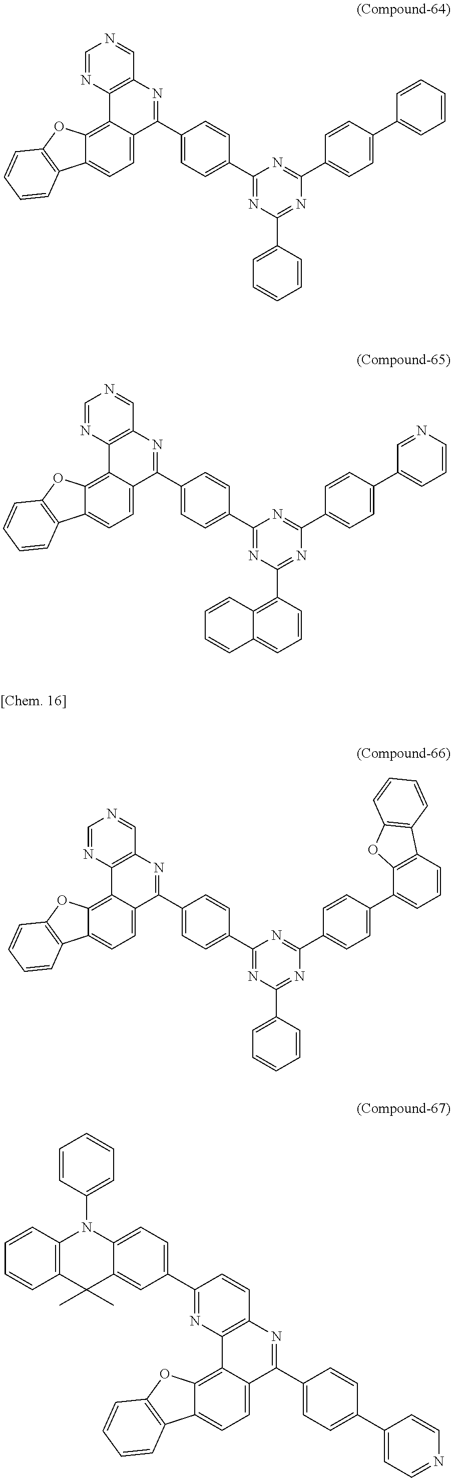

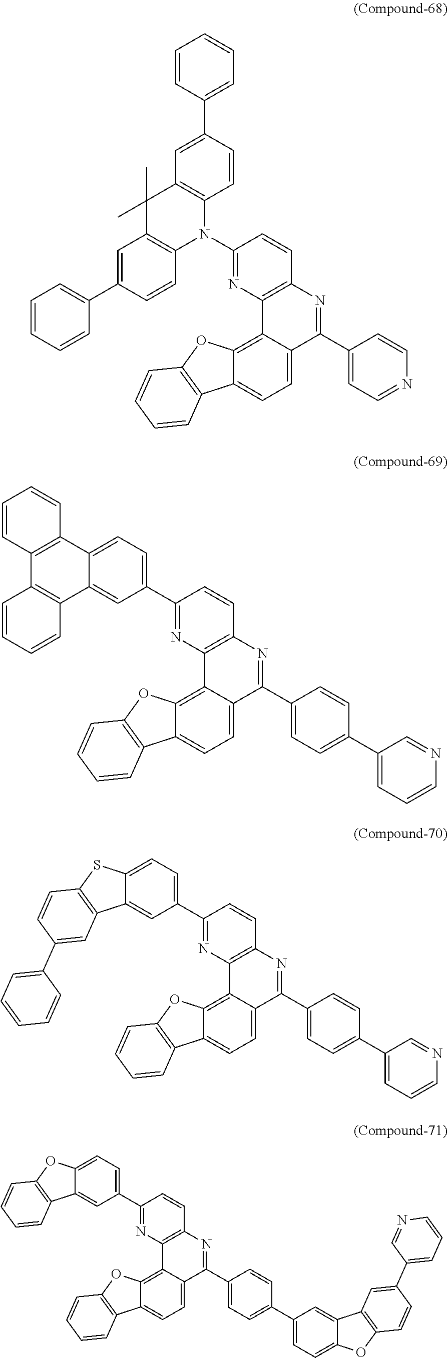

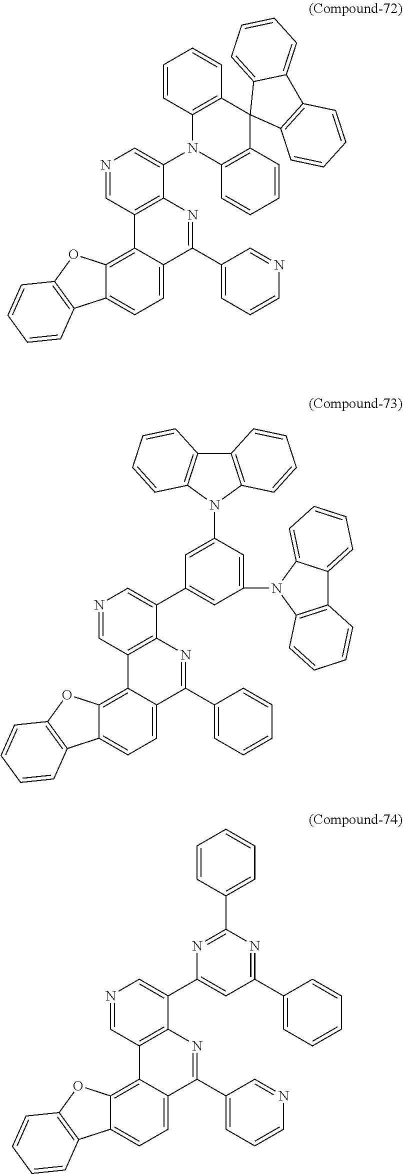

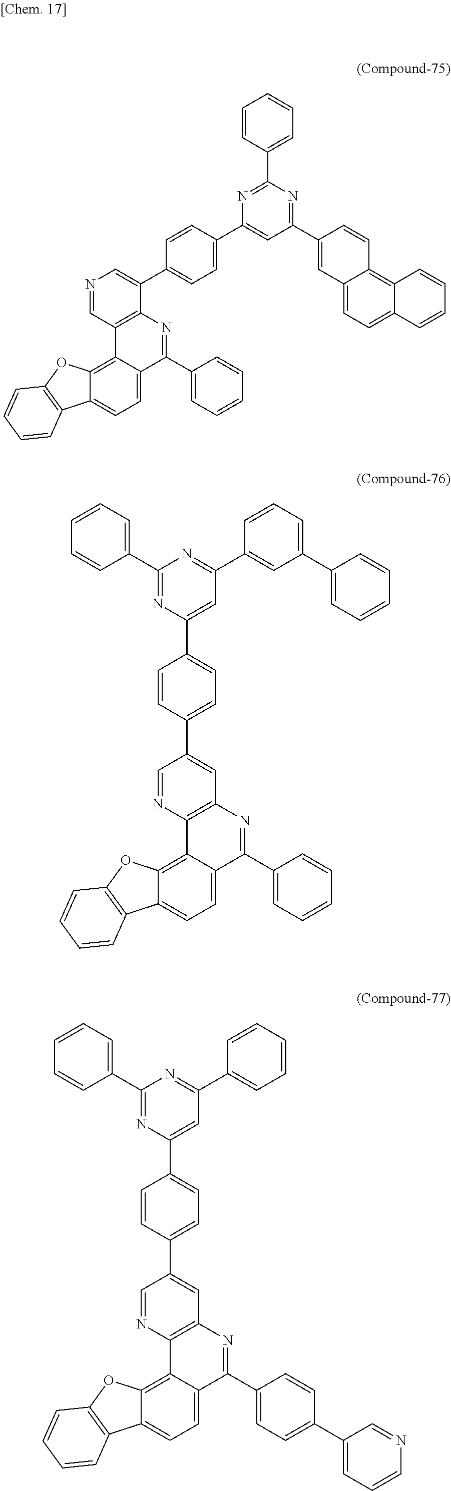

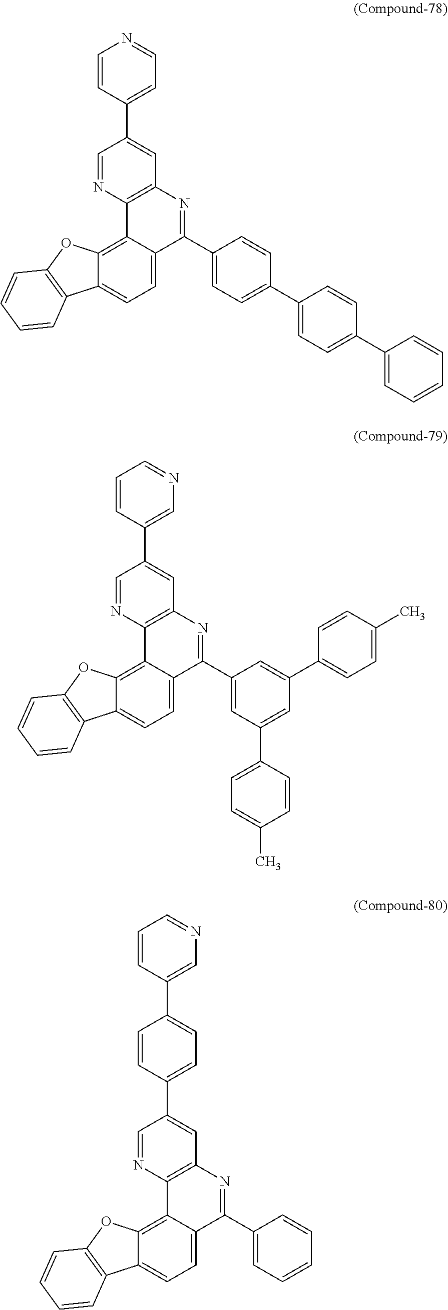

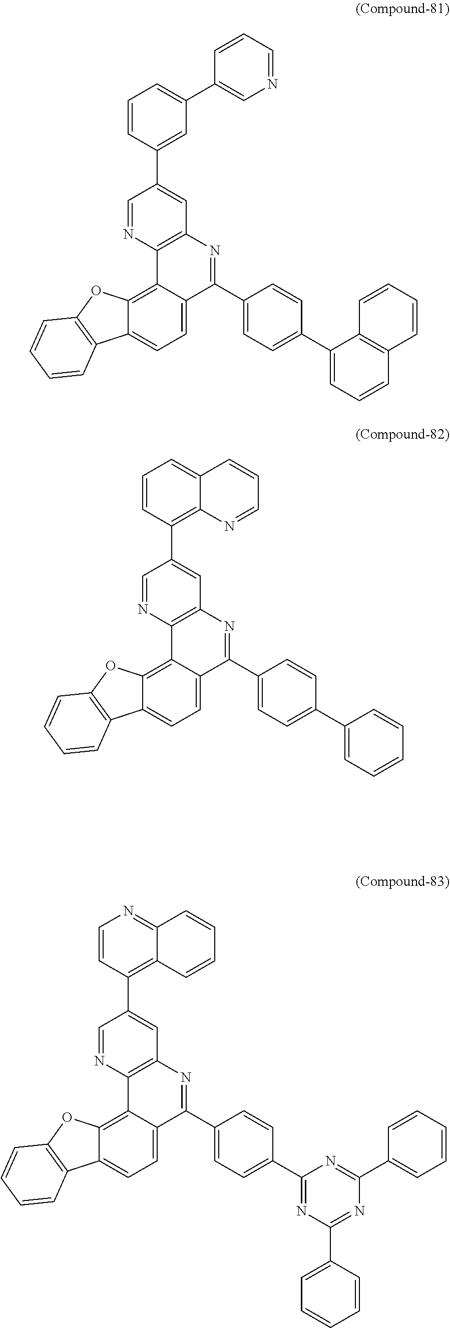

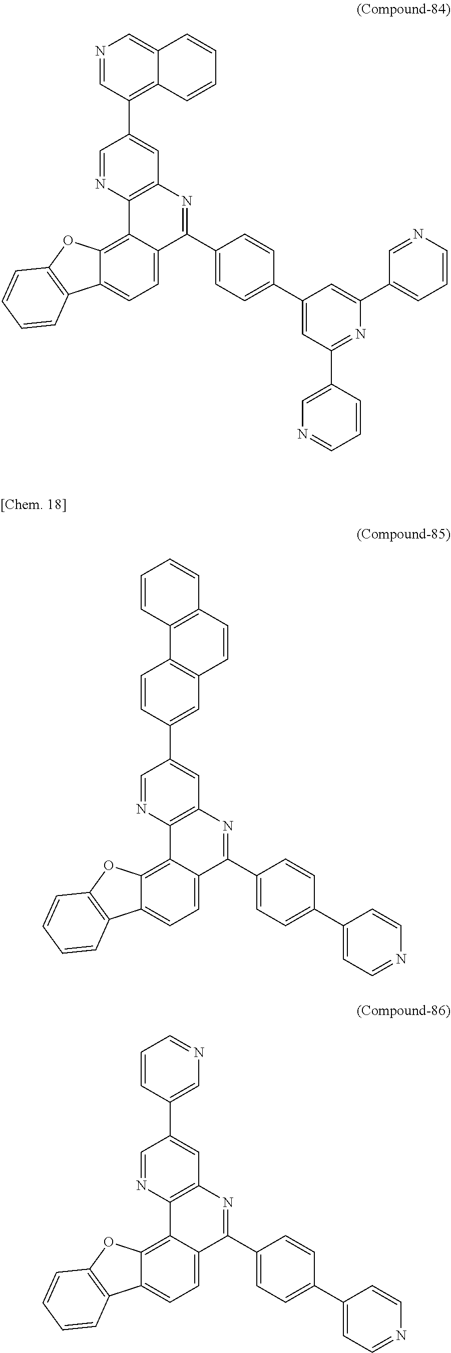

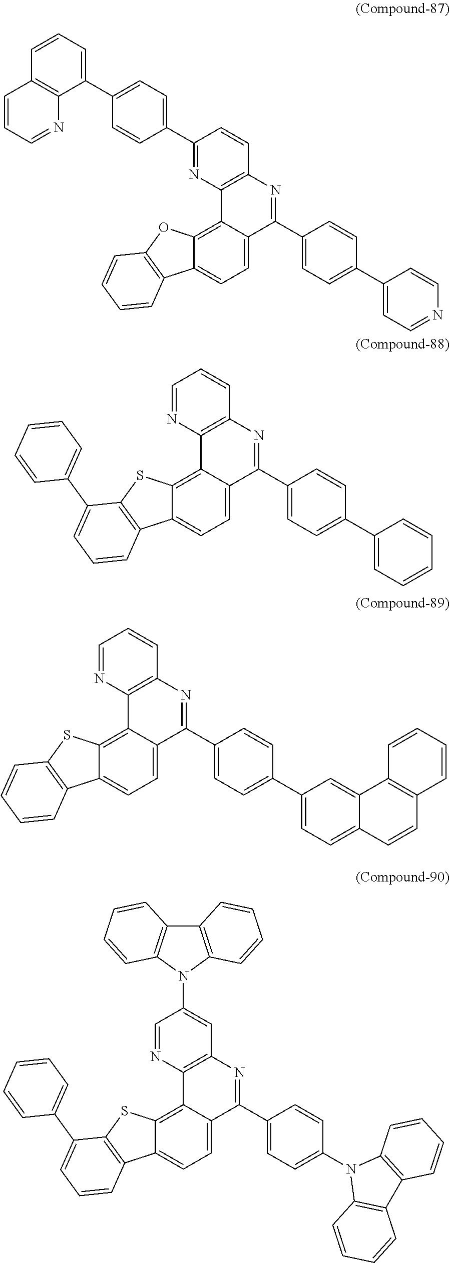

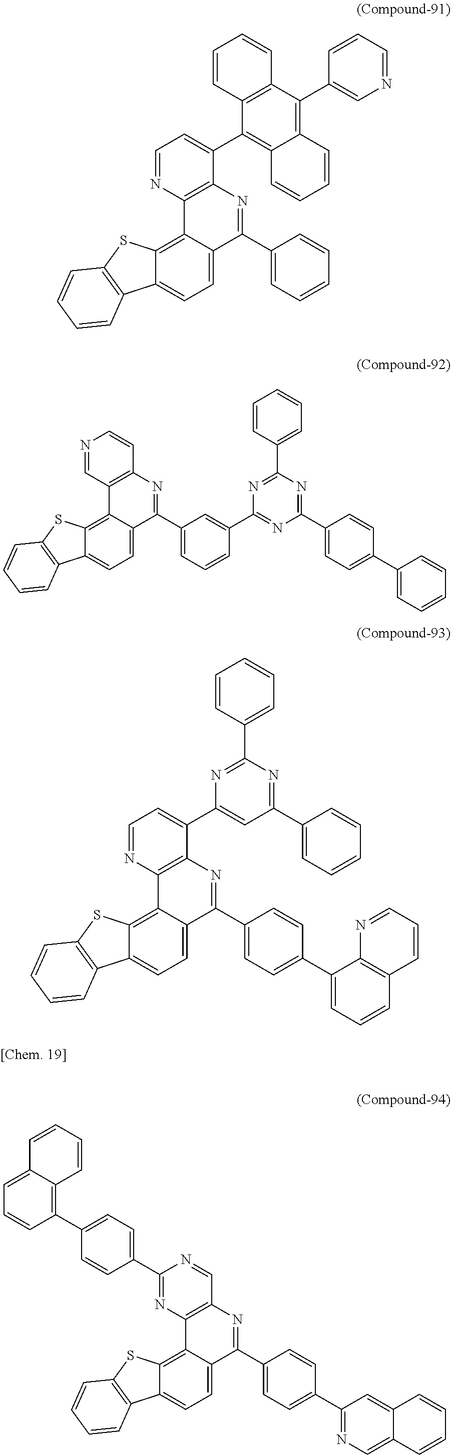

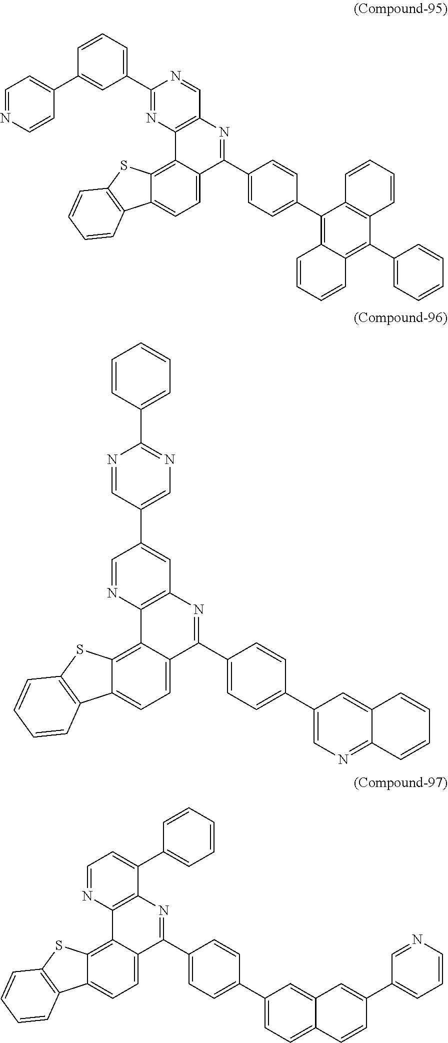

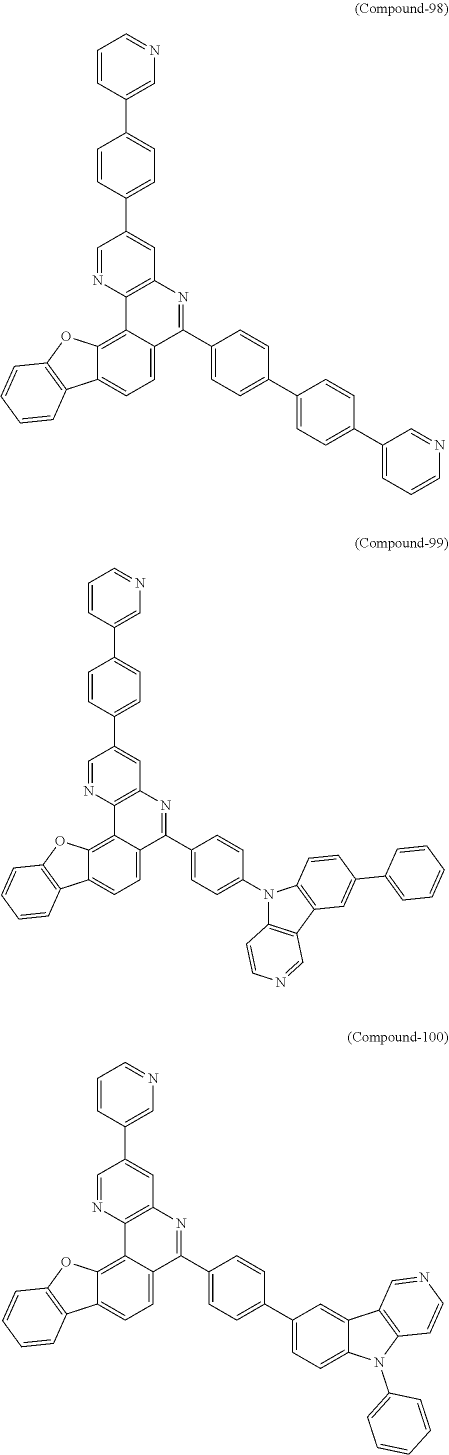

Purification of compounds that have an azaindeno [1,2-c] phenanthrene ring structure and are represented by the general formulae (A-1) to (A-6) was carried out by purification by column chromatography, adsorption purification with silica gel, activated carbon, activated clay, or the like, recrystallization with a solvent, a crystallization method, a sublimation purification method, or the like. Identification of the compounds was performed by NMR analysis. As physical property values, a melting point, a glass transition point (Tg), and a work function were measured. The melting point is an index of vapor deposition property. The glass transition point (Tg) is an index of a thin film stability. The work function is an index of a hole transport property and a hole blocking property.

The melting point and the glass transition point (Tg) were measured with a powder using a high sensitivity differential scanning calorimeter (DSC3100SA manufactured by Bruker AXS GmbH).

The work function was obtained by preparing a thin film of 100 nm on an ITO substrate and using an ionization potential measuring apparatus (PYS-202 manufactured by Sumitomo Heavy Industries, Ltd.).

Examples of the structure of the organic EL device according to the embodiment of the present disclosure include those including an anode, a hole injection layer, a hole transport layer, a light-emitting layer, an electron transport layer, an electron injection layer, and a cathode in the stated order on a substrate, those including an electron blocking layer between the hole transport layer and the light-emitting layer, and those including a hole blocking layer between the light-emitting layer and the electron transport layer. in the multilayer structures, several organic layers can be omitted or combined. For example, the hole injection layer and the hole transport layer may be combined or the electron injection layer and the electron transport layer may be combined. Further, two or more organic layers having the same function can be stacked. For example, two hole transport layers may be stacked, two light-emitting layers may be stacked, or two electron transport layers may be stacked.

As the anode of the organic EL device according to the embodiment of the present disclosure, an electrode material having a large work function such as ITO and gold is used.

As the hole injection layer of the organic EL device according to the embodiment of the present disclosure, an acceptor heterocyclic compound such as hexacyanoazatriphenylene or a coating type polymer material in addition to a porphyrin compound typified by copper phthalocyanine, a starburst type triphenylamine derivative, an arylamine compound having a structure in which two or more triphenylamine structures or carbazolyl structures are contained in the molecule and the structures are bonded to each other by a single bond or a divalent group containing no hetero atom, or the like can be used. These materials can be formed into a thin film by a known method such as a spin coat method and an ink jet method in addition to a vapor deposition method.

As the hole transport layer of the organic EL device according to the embodiment of the present disclosure, a benzidine derivative such as N,N′-diphenyl-N,N′-di(m-tolyl)-benzidine (hereinafter, referred to as TPD), N,N′-diphenyl-N,N′-di(α-naphthyl)-benzidine (hereinafter, referred to as NPD), and N,N,N′,N′-tetrabiphenylylbenzidine, 1,1-bis[(di-4-tolylamino)phenyl]cyclohexane (hereinafter, referred to as TAPC), an arylamine compound having a structure in which two or more triphenylamine structures or carbazolyl structures are contained in the molecule and the structures are bonded to each other by a single bond or a divalent group containing no hetero atom, e.g., N, N, N′, N′-tetrabiphenylylbenzidine, or the like can be used. These materials may be deposited alone. However, any of the materials may be mixed with another material and used as a single deposited layer. Further, a stacked structure may be achieved by depositing layers of the plurality of materials alone, or mixing the plurality of materials and depositing layers thereof. Alternatively, a stacked structure of at least one layer of any of the plurality of materials deposited alone and at least one layer obtained by mixing the plurality of materials and depositing at least one layer thereof may be achieved. Further, as a hole injection/transport layer, a coating polymer material such as poly (3,4-ethylenedioxythiophene) (hereinafter, referred to as PEDOT)/poly(styrene sulfonate) (hereinafter, referred to as PSS) can be used. These materials can be formed into a thin film by a known method such as a spin coat method and an ink jet method in addition to a vapor deposition method.

Further, for the hole injection layer or hole transport layer, those obtained by P-doping the material typically used for the layer with trisbromophenylamine hexachloroantimony or a radialene derivative (see, for example, WO 2014/009310) , a polymer compound having, as a partial structure, the structure of a benzidine derivative such as TPD, or the like can be used.

For the electron blocking layer of the organic EL device according to the embodiment of the present disclosure, a compound having an electron blocking property, such as a carbazol derivative such as 4,4′,4″-tri(N-carbazolyl) triphenylamine (hereinafter, referred to as TCTA), 9,9-bis[4-(carbazol-9-yl)phenyl]fluorene, 1,3-bis(carbazol-9-yl)benzene (hereinafter, referred to as mCP), and 2,2-bis(4-carbazol-9-ylphenyl)adamantane (hereinafter, referred to as Ad-Cz), and a compound having a triphenylsilyl group and a triarylamine structure typified by 9-[4-(carbazol-9-yl)phenyl]-9-[4-(triphenylsilyl)phenyl]-9H-fluorene, can be used. These materials may be deposited alone. However, any of the materials may be mixed with another material and used as a single deposited layer. Further, a stacked structure may be achieved by depositing layers of the plurality of materials alone, or mixing the plurality of materials and depositing layers thereof. Alternatively, a stacked structure of at least one layer of any of the plurality of materials deposited alone and at least one layer obtained by mixing the plurality of materials and depositing at least one layer thereof may be achieved. These materials can be formed into a thin film by a known method such as a spin coat method and an ink jet method in addition to a vapor deposition method.

For the light-emitting layer of the organic EL device according to the embodiment of the present disclosure, a metal complex of a quinolinol derivative including Alq₃, various metal complexes, an anthracene derivative a bis-styryl benzene derivative, a pyrene derivative, an oxazole derivative, a poly(p-phenylene vinylene) derivative, or the like in addition to the compound having an azaindeno [1,2-c] phenanthrene ring structure according to the embodiment of the present disclosure can be used. Further, the light-emitting layer may be formed of a host material and a dopant material. As the host material, an anthracene derivative is favorably used. In addition, not only the above-mentioned light-emitting material including the compound having an azaindeno [1,2-c] phenanthrene ring structure according to the embodiment of the present disclosure but also a heterocyclic compound having an indole ring as a partial structure of the fused ring, a heterocyclic compound having a carbazol ring as a partial structure of the fused ring, a carbazol derivative, a thiazole derivative, a benzimidazole derivative, a polydialkylfluorene derivative, or the like can be used. Further, as the dopant material, quinacridone, coumarin, rubrene, perylene, and dervatives thereof, a benzopyran derivative, a rhodamine derivative, an aminostyryl derivative, or the like can be used. These materials may be deposited alone. However, any of the materials may be mixed with another material and used as a single deposited layer. Further, a stacked structure may be achieved by depositing layers of the plurality of materials alone, or mixing the plurality of materials and depositing layers thereof. Alternatively, a stacked structure of at least one layer of any of the plurality of materials deposited alone and at least one layer obtained by mixing the plurality of materials and depositing at least one layer thereof may be achieved.

Further, as the light-emitting material, a phosphorescent material can be used. As the phosphorescent material, a phosphorescent material of a metal complex such as iridium and platinum can be used. A green phosphorescent material such as Ir(ppy)₃, a blue phosphorescent material such as Flrpic and FIr6, a red phosphorescent material such as Btp₂Ir (acac), or the like is used. As the host material (having a hole injection/transporting property) at this time, a compound having a pyridoindole ring structure in addition to 4,4′-di(N-carbazolyl) biphenyl (hereinafter, referred to as CBP) and a carbazol derivative such as TCTA and mCP can be used. As a host material having an electron transportability, p-bis(triphenylsilyl)benzene (hereinafter, referred to as UGH2), 2,2′,2″-(1,3,5-phenylene)-tris(1-phenyl-1H-benzimidazole) (hereinafter, referred to as, TPBI), the compound having an azaindeno [1,2-c] phenanthrene ring structure according to the embodiment of the present disclosure, or the like can be used, and an organic EL device having high performance can be prepared.

In order to avoid concentration quenching, it is favorable to dope the host material with the phosphorescent material by co-deposition in the range of 1 to 30 weight percent with respect to the entire light-emitting layer.

Further, as the light-emitting material, a material emitting delayed fluorescence such as a CDCB derivative including PIC-TRZ, CC2TA, PXZ-TRZ, and 4CzIPN can be used (see, for example, Appl. Phys. Let., 98,083302(2011)). These materials can be formed into a thin film by a known method such as a spin coat method and an ink jet method in addition to a vapor deposition method.

For the hole blocking layer of the organic EL device according to the embodiment of the present disclosure, a compound having a hole blocking property, such as a phenanthroline derivative such as BCP, a metal complex of a quinolinol derivative such as aluminum (III) bis(2-methyl-8-quinolinate)-4-phenylphenolate (hereinafter, referred to as BAlq), various rare earth complexes, an oxazole derivative, a triazole derivative, a triazine derivative, a pyrimidine derivative, an oxadiazole derivative, and a benzoazole derivative in addition to the compound having an azaindeno [1,2-c] phenanthrene ring structure according to the embodiment of the present disclosure can be used. These materials may double as the material of the electron transport layer. These materials may be deposited alone. However, any of the materials may be mixed with another material and used as a single deposited layer. Further, a stacked structure may be achieved by depositing layers of the plurality of materials alone, or mixing the plurality of materials and depositing layers thereof. Alternatively, a stacked structure of at least one layer of any of the plurality of materials deposited alone and at least one layer obtained by mixing the plurality of materials and depositing at least one layer thereof may be achieved. These materials can be formed into a thin film by a known method such as a spin coat method and an ink jet method in addition to a vapor deposition method.

As the electron transport layer of the organic EL device according to the embodiment of the present disclosure, a metal complex of a quinolinol derivative including Alq₃ and BAlq, various metal complexes, a triazole derivative, triazine derivative, a pyrimidine derivative, an oxadiazole derivative, a pyridine derivative, a benzimidazole derivative, a benzoazole derivative, a thiadiazole derivative, an anthracene derivative, a carbodiimide derivative, a quinoxaline derivative, a pyridoindole derivative, a phenanthroline derivative, a silole derivative, or the like in addition to the compound having an azaindeno [1,2-c] phenanthrene ring structure according to the embodiment of the present disclosure can be used. These materials may be deposited alone. However, any of the materials may be mixed with another material and used as a single deposited layer. Further, a stacked structure may be achieved by depositing layers of the plurality of materials alone, or mixing the plurality of materials and depositing layers thereof. Alternatively, a stacked structure of at least one layer of any of the plurality of materials deposited alone and at least one layer obtained by mixing the plurality of materials and depositing at least one layer thereof may be achieved. These materials can be formed into a thin film by a known method such as a spin coat method and an ink jet method in addition to a vapor deposition method.

For the electron injection layer of the organic EL device according to the embodiment of the present disclosure, an alkali metal salt such as lithium fluoride and cesium fluoride, an alkaline earth metal salt such as magnesium fluoride, a metal complex of a quinolinol derivative such as lithiumquinolinol, a metal oxide such as an aluminum oxide, a metal such as ytterbium (Yb), samarium (Sm), calcium (Ca), strontium (Sr), and cesium (Cs), or the like in addition to the compound having an azaindeno [1,2-c] phenanthrene ring structure according to the embodiment of the present disclosure can be used. However, this can be omitted in the favorable selection of the electron transport layer and the cathode.

Further, for the electron injection layer or electron transport layer, those obtained by N-doping the material typically used for the layer with a metal such as cesium can be used.

In the cathode of the organic EL device according to the embodiment of the present disclosure, an electrode material having a low work function, such as aluminum, an alloy having a lower work function, such as a magnesium silver alloy, a magnesium calcium alloy, a magnesium indium alloy, and an aluminum magnesium alloy, ITO, IZO, or the like is used as the electrode material.

Hereinafter, the embodiment of the present disclosure will be specifically described by way of Examples. However, the present disclosure is not limited to the following Examples as long as the essence of the present disclosure is not exceeded.

### EXAMPLE 1

### Synthesis of 6-{4-(10-phenyl-anthracen-9-yl)phenyl}-13-oxa-1,5-diazaindeno [1,2-c] phenanthrene (compound-6)

A reaction vessel was charged with 15.0 g of 2-chloro-3-nitropyridine, 21.0 g of dibenzofuran-4-boronic acid, 105 mL of toluene, and 26 mL of ethanol. Subsequently, an aqueous solution obtained by previously dissolving 15.7 g of potassium carbonate in 56 mL of H₂O was added thereto, and nitrogen gas was bubbled for 30 minutes while being irradiated with ultrasonic waves. Two point two g of tetrakistriphenylphosphine palladium was added thereto, and the obtained mixture was stirred overnight under heating reflux. After cooling the mixture, ethyl acetate was added to the system to perform an extraction operation, and the organic layer was concentrated to obtain a crude product. By adding n-heptane to this crude product and filtering out the precipitated solid, 27.5 g (yield 100%) of 2-(dibenzofuran-4-yl)-3-nitropyridine (intermediate-1) was obtained.

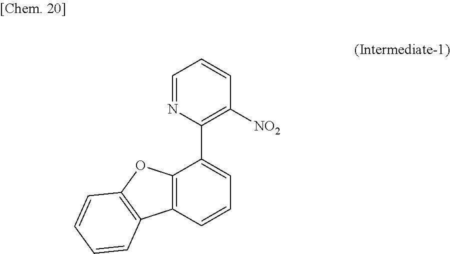 [SMILES: O=[N+]([O-])c1cccnc1-c1cccc2c1oc1ccccc12]

Subsequently, the reaction vessel was charged with 27.5 g of 2-(dibenzofuran-4-yl)-3-nitropyridine (intermediate-1), 24.9 g of iron powder, and 410 ml of ethanol. Two hundred ml of ammonium chloride water was added thereto, and the obtained mixture was stirred overnight under heating reflux. After cooling the mixture, dichloromethane was added to the filtrate obtained by filtration to perform an extraction operation, and the organic layer was concentrated to obtain 22.5 g (yield 92%) of 3-amino-2-(dibenzofuran-4-yl)-pyridine (intermediate-2).

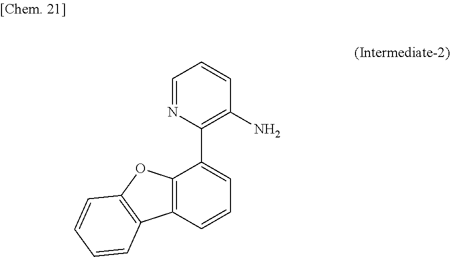 [SMILES: Nc1cccnc1-c1cccc2c1oc1ccccc12]

Subsequently, the reaction vessel was charged with 22.5 g of 3-amino-2-(dibenzofuran-4-y1)-pyridine (intermediate-2), 9.6 g of triethylamine, and 225 ml of dichloromethane, and a solution obtained by previously dissolving 16.6 g of 4-chlorobenzoyl chloride in 160 ml of dichloromethane was added dropwise to the system stirred with ice cooling. After raising the reaction temperature to room temperature, the mixture was stirred overnight. H₂O was added to the reaction system to perform an extraction operation, and the organic layer was concentrated to obtain a crude product. By adding n-heptane to this crude product and filtering out the precipitated solid, 34.0 g (yield 98%) of 4-chloro-N-{2-(dibenzofuran-4-yl)-pyridin-3-yl}-benzamide (intermediate-3) was obtained.

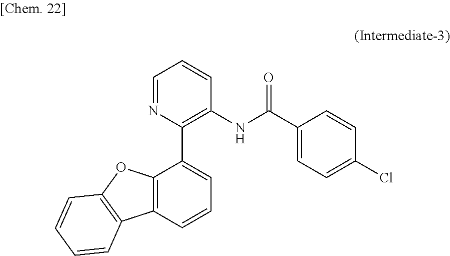 [SMILES: O=C(Nc1cccnc1-c1cccc2c1oc1ccccc12)c1ccc(Cl)cc1]

Subsequently, the reaction vessel was charged with 15.0 g of 4-chloro-N-{2-(dibenzofuran-4-yl)-pyridin-3-yl}-benzamide (intermediate-3), 17.3 g of phosphoryl chloride, and 1,2-dichlorobenzene. Two ml of 12 N hydrochloric acid was added thereto, and the mixture was stirred overnight at 140° C. After cooling the mixture, an NaOH aqueous solution was added for neutralization. An extraction operation was performed, and the organic layer was concentrated to obtain a crude product. By adding methanol to this crude product and filtering out the precipitated solid, 6.0 g (yield 42%) of 6-(4-chloro-phenyl)-13-oxa-1,5-diazaindeno [1,2-c] phenanthrene (intermediate-4) was obtained.

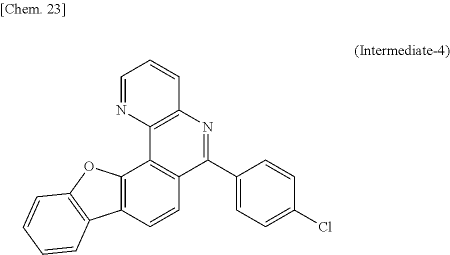 [SMILES: Clc1ccc(-c2nc3cccnc3c3c2ccc2c4ccccc4oc23)cc1]

Subsequently, the reaction vessel was charged with 8.1 g of 6-(4-chloro-phenyl)-13-oxa-1,5-diazaindeno [1,2-c] phenanthrene (intermediate-4), 7.6 g of 9-(10-phenyl) anthraceneboronic acid, 56 mL of toluene, and 14 mL of ethanol. Subsequently, an aqueous solution obtained by previously dissolving 5.9 g of potassium carbonate in 21 mL of H₂O was added thereto, and nitrogen gas was bubbled for 30 minutes while being irradiated with ultrasonic waves. Zero point seven g of tetrakistriphenylphosphine palladium was added thereto, and the mixture was stirred for two days under heating reflux. After cooling the mixture, ethyl acetate was added to the system to perform an extraction operation, and the organic layer was concentrated to obtain a crude product. This crude product was purified by column chromatograpy using dichloromethane/ethyl acetate mixed solvent to obtain 4.2 g (yield 33%) of a yellow powder of 6-{4-(10-phenyl-anthracen-9-yl)phenyl}-13-oxa-1,5-diazaindeno [1,2-c] phenanthrene(compound-6).

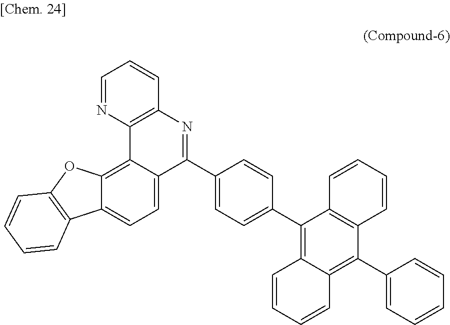 [SMILES: c1ccc(-c2c3ccccc3c(-c3ccc(-c4nc5cccnc5c5c4ccc4c6ccccc6oc45)cc3)c3ccccc23)cc1]

The structure of the obtained yellow powder was identified using NMR.

The following 26 hydrogen signals were detected by ¹H-NMR (CDCl₃). δ(ppm)=9.39(1H), 8.74(1H), 8.46(2H), 8.22(1H), 8.11(2H), 8.09(1H), 7.95(2H), 7.88(1H), 7.82-7.74(4H), 7.72-7.59(4H), 7.55(3H), 7.44(4H).

### EXAMPLE 2

### Synthesis of 6-{4-(pyridin-3-yl)phenyl}-13-oxa-1,5-diazaindeno [1,2-c] phenanthrene (compound-13)

A reaction vessel was charged with 6.0 g of 6-(4-chloro-phenyl)-13-oxa-1,5-diazaindeno [1,2-c] phenanthrene (intermediate-4), 2.3 g of 3-pyridylboronic acid, 0.3 g of bis(dibenzylideneacetone) palladium(0), 0.4 g of tricyclohexyl phosphine, and 10.0 g of tripotassium phosphate. The mixture was stirred under reflux overnight in a 1,4-dioxane/H₂O mixed solvent.

After cooling the mixture, methanol was added and the precipitated solid was collected to obtain a crude product. This crude product was purified by recrystallization using monochlorobenzene to obtain 3.2 g (yield 47%) of a white powder of 6-{4-(pyridine-3-yl)phenyl}-13-oxa-1,5-diazaindeno [1,2-c] phenanthrene (compound-13).

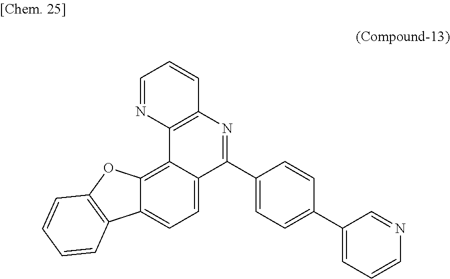 [SMILES: c1cncc(-c2ccc(-c3nc4cccnc4c4c3ccc3c5ccccc5oc34)cc2)c1]

The structure of the obtained white powder was identified using NMR.

The following 17 hydrogen signals were detected by ¹H-NMR (CDCl₃).

δ(ppm)=9.35(1H), 9.01(1H), 8.69(1H), 8.62(1H), 8.28(2H), 8.16(1H), 8.05(1H), 8.03(1H), 7.91(4H), 7.83(1H), 7.66(1H), 7.51(1H), 7.47(1H).

### EXAMPLE 3

The melting point and the glass transition point of the compound having an azaindeno [1,2-c] phenanthrene ring structure represented by the general formula (A-1) were measured by a high sensitivity differential scanning calorimeter (DSC3100SA manufactured by Bruker AXS GmbH).

| | Melting point | Glass transition point |
|---|---|---|
| Compound of Example 1 | 364° C. | 184° C. |
| Compound of Example 2 | 278° C. | 111° C. |

The compound having an azaindeno [1,2-c] phenanthrene ring structure represented by the general formula (A-1) has the glass transition point of not less than 100° C., which shows that the thin film state is stable.

### EXAMPLE 4

The compound having an azaindeno [1,2-c] phenanthrene ring structure represented by the general formula (A-1) was used to prepare a vapor deposition film having a film thickness of 100 nm on an ITO substrate, and the work function thereof was measured by an ionization potential measuring apparatus(PYS-202 manufactured by Sumitomo Heavy Industries, Ltd.).

| | Work function |
|---|---|
| Compound of Example 1 | 6.02 eV |
| Compound of Example 2 | 6.62 eV |

The compound having an azaindeno [1,2-c] phenanthrene ring structure represented by the general formula (A-1) exhibits a suitable energy level as compare with a work function of 5.8 to 6.0 eV of the general electron transport material such as Alq₃, and it can be seen that the compound has a favorable electron transport ability.

### EXAMPLE 5

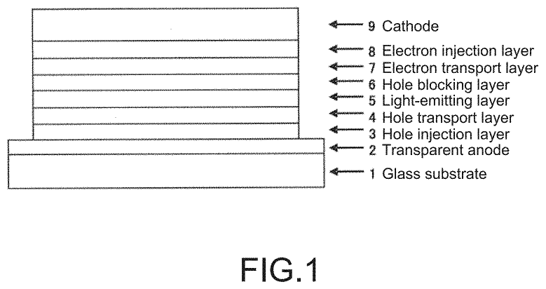

The organic EL device was prepared by depositing a hole injection layer 3, a hole transport layer 4, a light-emitting layer 5, a hole blocking layer 6, an electron transport layer 7, an electron injection layer 8, and a cathode (aluminum electrode) 9 in the stated order on a transparent anode 2, which has been formed on a glass substrate 1 as an ITO electrode in advance, as shown in FIG. 1.

Specifically, after performing, in isopropyl alcohol for 20 minutes, ultrasonic leaning on the glass substrate 1 on which ITO having a film thickness of 50 nm was formed, the glass substrate 1 was dried for 10 minutes on a hot plate heated to 200° C. After that, UV ozone treatment was performed for 15 minutes, and then, the ITO-attached glass substrate was mounted in a vacuum deposition machine. The pressure in the vacuum deposition machine was reduced to not more than 0.001 Pa. Subsequently, a film of an electron acceptor (Acceptor-1) having the following structural formula and a compound (HTM-1) having the following structural formula were formed, as the hole injection layer 3, to have a film thickness of 10 nm and cover the transparent anode 2 by binary deposition at a deposition rate in which the ratio of the evaporation rates of Acceptor-1 and HTM-1 was 3:97. As the hole transport layer 4, a film of the compound (HTM-1) having the following structural formula was formed on the hole injection layer 3 to have a film thickness of 60 nm. A film of a compound (EMD-1) having the following structural formula and a compound (EMH-1) having the following structural formula were formed, as the light-emitting layer 5, on the hole transport layer 4 to have a film thickness of 20 nm by binary deposition at a deposition rate in which the ratio of the evaporation rates of EMD-1 and EMH-1 was 5:95. A film of the compound (compound-6) according to Example 1 of the present disclosure and a compound (ETM-1) having the following structural formula were formed, as the hole blocking layer/electron transport layer 6 and 7, on the light-emitting, layer 5 to have a film thickness of 30 nm by binary deposition at a deposition rate in which the ratio of the evaporation rates of the compound-6 according to Example 1 of the present disclosure and ETM-1 was 50:50. A film of lithium fluoride was formed, as the electron injection layer 8, on the hole blocking layer/electron transport layer 6 and 7 to have a film thickness of 1 nm. Finally, aluminum was deposited to have a thickness of 100 nm to form the cathode 9. The characteristics of the prepared organic EL device were measured at room temperature in the atmosphere. The measurement results of the light-emitting characteristics when a direct current voltage was applied to the prepared organic EL device were collectively shown in Table 1.

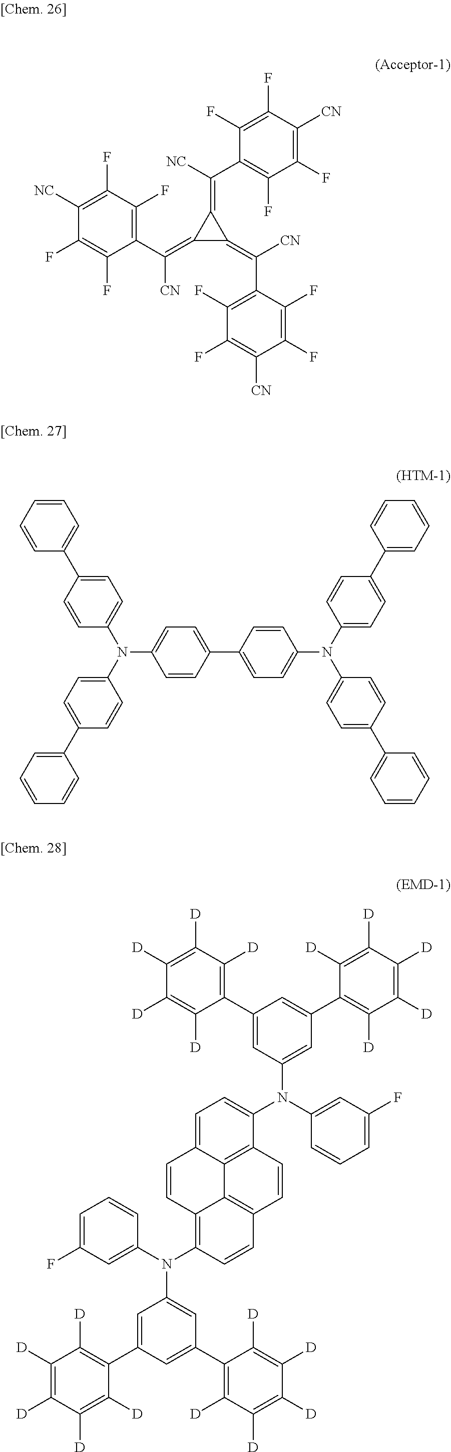 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; [2H]c1c([2H])c([2H])c(-c2cc(-c3c([2H])c([2H])c([2H])c([2H])c3[2H])cc(N(c3cccc(F)c3)c3ccc4ccc5c(N(c6cccc(F)c6)c6cc(-c7c([2H])c([2H])c([2H])c([2H])c7[2H])cc(-c7c([2H])c([2H])c([2H])c([2H])c7[2H])c6)ccc6ccc3c4c65)c2)c([2H])c1[2H]]

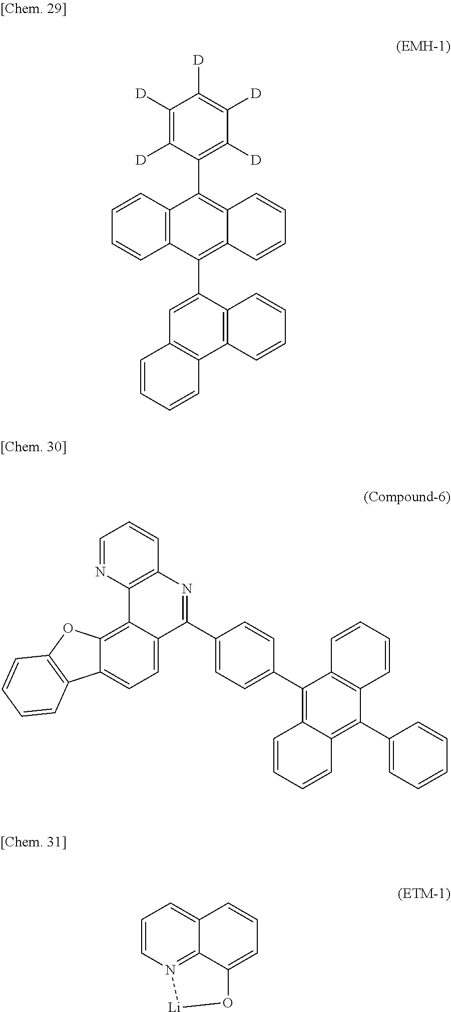

### EXAMPLE 6

An organic EL device was prepared under the same conditions except that the compound (compound-13) according to Example 2 was used instead of the compound (compound-6) according to Example 1 of the present disclosure as the material of the hole blocking layer/electron transport layer 6 and 7 in Example 5 and binary deposition was performed at a deposition rate in which the ratio of the evaporation rates of the (compound-13) and ETM-1 was 50:50. The characteristics of the prepared organic EL device were measured at room temperature in the atmosphere. The measurement results of the light-emitting characteristics when a direct current voltage was applied to the prepared organic EL device were collectively shown in Table 1.

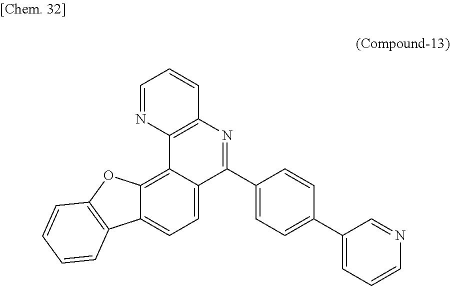 [SMILES: c1cncc(-c2ccc(-c3nc4cccnc4c4c3ccc3c5ccccc5oc34)cc2)c1]

### COMPARATIVE EXAMPLE 1

For comparison, an organic EL device was prepared under the same conditions except that a compound (ETM-2) having the following structural formula (see, for example, WO 2010/074422) was used instead of the compound (compound-6) according to Example 1 of the present disclosure as the material of the hole blocking layer/electron transport layer 6 and 7 in Example 5 and binary deposition was performed at a deposition rate in which the ratio of the evaporation rates of ETM-2 and ETM-1 was 50:50. The characteristics of the prepared organic EL device were measured at room temperature in the atmosphere. The measurement results of the light-emitting characteristics when a direct current voltage was applied to the prepared organic EL device were collectively shown in Table 1.

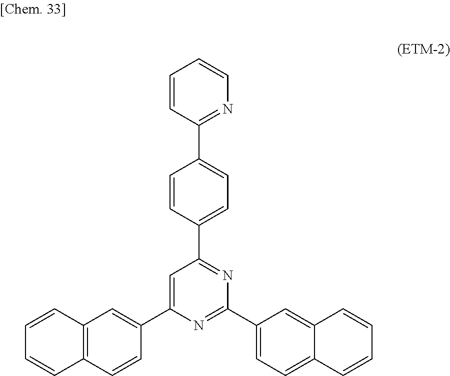 [SMILES: c1ccc(-c2ccc(-c3cc(-c4ccc5ccccc5c4)nc(-c4ccc5ccccc5c4)n3)cc2)nc1]

### COMPARATIVE EXAMPLE 2

For comparison, an organic EL device was prepared under the same conditions except that a compound (ETM-3) having the following structural formula (see, for example, WO 2017/111439) was used instead of the compound (compound-6) according to Example 1 of the present disclosure as the material of the hole blocking layer/electron transport layer 6 and 7 in Example 5 and binary deposition was performed at a deposition rate in which the ratio of the evaporation rates of ETM-3 and ETM-1 was 50:50. The characteristics of the prepared organic EL device were measured at room temperature in the atmosphere. The measurement results of the light-emitting characteristics when a direct current voltage was applied to the prepared organic EL device were collectively shown in Table 1.

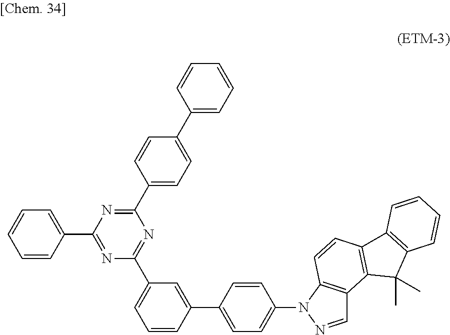 [SMILES: CC1(C)c2ccccc2-c2ccc3c(cnn3-c3ccc(-c4cccc(-c5nc(-c6ccccc6)nc(-c6ccc(-c7ccccc7)cc6)n5)c4)cc3)c21]

### COMPARATIVE EXAMPLE 3

For comparison, an organic EL device was prepared under the same conditions except that a compound (ETM-4) having the following structural formula (see, for example, WO 2017/111439) was used instead of the compound (compound-6) according to Example 1 of the present disclosure as the material of the hole blocking layer/electron transport layer 6 and 7 in Example 5 and binary deposition was performed at a deposition rate in which the ratio of the evaporation rates of ETM-4 and ETM-1 was 50:50. The characteristics of the prepared organic EL device were measured at room temperature in the atmosphere. The measurement results of the light-emitting characteristics when a direct current voltage was applied to the prepared organic EL device were collectively shown in Table 1.

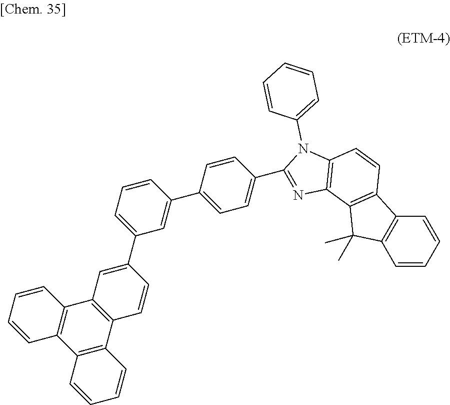 [SMILES: CC1(C)c2ccccc2-c2ccc3c(nc(-c4ccc(-c5cccc(-c6ccc7c8ccccc8c8ccccc8c7c6)c5)cc4)n3-c3ccccc3)c21]

The device lifetime was measured using each of the organic EL devices prepared in Examples 5 and 6 and Comparative Examples 1 to 3, and the results were collectively shown in Table 1. The device lifetime was measured as the time until the light emission luminance attenuated to 1900 cd/m² (corresponding to 95% in the case where the initial luminance was 100%: 95% attenuation) when constant current driving was performed with the light emission luminance (initial luminance) at the start of light emission set to 2000 cd/m².

TABLE 1

| | Hole blocking | | Luminance | Light emission | Power efficiency | |
|---|---|---|---|---|---|---|
| | layer/electron | Voltage[V] | [cd/m2] | efficiency [cd/A] | [lm/W] | Device lifetime |
| | transport layer | (@10 mA/cm2) | (@10 mA/cm2) | (@10 mA/cm2) | (@10 mA/cm2) | 95% attenuation |
| Example 5 | Compound 6/ | 3.47 | 891 | 8.93 | 8.09 | 127 hours |
| | ETM-1 | | | | | |
| Example 6 | Compound-13/ | 3.55 | 897 | 8.98 | 7.96 | 115 hours |
| | ETM-1 | | | | | |
| Comparative | ETM-2/ | 3.66 | 753 | 7.53 | 6.45 | 43 hours |
| Example 1 | ETM-1 | | | | | |
| Comparative | ETM-3/ | 3.76 | 695 | 6.95 | 5.80 | 65 hours |
| Example 2 | ETM-1 | | | | | |
| Comparative | ETM-4/ | 3.81 | 762 | 7.62 | 6.28 | 63 hours |
| Example 3 | ETM-1 | | | | | |

As shown in Table 1, the drive voltage when a current having a current density of 10 mA/cm² was caused to flow was lowered to 3.47 to 3.55 V in the organic EL devices according to Examples 5 and 6 as compared with the 3.66 to 3.81 V of the organic EL devices according to Comparative Examples 1 to 3 using the compounds ETM-2 to 4 having the above-mentioned structural formulae, respectively. Further, the light emission efficiency was improved to 8.93 to 8.98 cd/A in the organic EL devices according to Examples 5 and 6 as compared with 6.95 to 7.62 cd/A of the organic EL devices according to Comparative Examples 1 to 3. Also the power efficiency of the organic EL devices according to Examples 5 and 6 was improved to 7.96 to 8.09 lm/W as compared with 5.80 to 6.45 lm/W of the organic EL devices according to Comparative Examples 1 to 3. In particular, the device lifetime (95% attenuation) was largely extended to 115 to 127 hours in the organic EL devices according to Examples 5 and 6 as compared with 43 to 65 hours of the organic EL devices according to Comparative Examples 1 to 3.

As described above, the organic EL device according to the embodiment of the present disclosure is excellent in the light emission efficiency and power efficiency as compared with the devices using the compounds (ETM-2˜4) having the above-mentioned structural formulae. Thus, it has been found that it is possible to realize an organic EL device having a long lifetime.

### INDUSTRIAL APPLICABILITY

The compound having a specific azaindeno [1,2-c] phenanthrene ring structure according to an embodiment of the present disclosure is excellent in electron injection property and electron transport ability, and has a stable thin film, and thus is suitably used as a compound for organic EL device. By preparing an organic EL device using the compound, it is possible to achieve a high efficiency, reduce the drive voltage, and improve the durability. For example, it has become possible to expand to home appliances and lighting applications.

What is claimed is:

1. A compound having an azaindeno phenanthrene ring structure, the compound being represented by the following general formula (A-1). 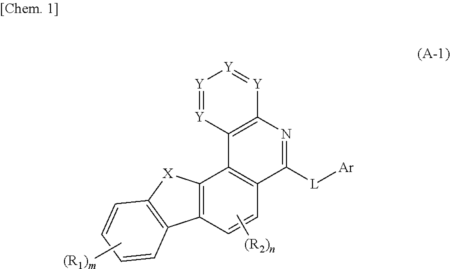
(wherein, Ys may be the same as or different from each other, and each represent a hydrogen atom, a deuterium atom, a fluorine atom, a chlorine atom, a cyano group, a nitro group, a trimethylsilyl group, a carbon atom having a triphenylsilyl group, a carbon atom having a substituted or unsubstituted aromatic hydrocarbon group, a carbon atom having a substituted or unsubstituted aromatic heterocyclic group, a carbon atom having a substituted or unsubstituted fused polycyclic aromatic group, a carbon atom having a linear or branched alkyl group having 1 to 6 carbon atoms which may have a substituent, a carbon atom having a cycloalkyl group having 5 to 10 carbon atoms which may have a substituent, a carbon atom having a linear or branched alkenyl group having 2 to 6 carbon atoms which may have a substituent, a carbon atom having a linear or branched alkyloxy group having 1 to 6 carbon atoms which may have a substituent, a carbon atom having a cycloalkyloxy group having 5 to 10 carbon atoms which may have a substituent, or a nitrogen atom,
L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁ and R₂ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,
X represents an oxygen atom or a sulfur atom,
m represents an integer of 0 to 4, and
n represents an integer of 0 to 2.
However, where m or n is an integer of two or more, at least one of Ys may be a nitrogen atom, and a plurality of R₁ or a plurality of R₂, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

2. The compound having an azaindeno phenanthrene ring structure according to claim 1,
wherein
the general formula (A-1) is represented by the following general formula (A-2). 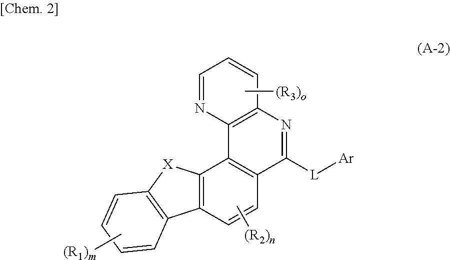
(wherein, L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁, R₂, and R₃ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,
X represents an oxygen atom or a sulfur atom,
m represents an integer of 0 to 4,
n represents an integer of 0 to 2, and
o represents an integer of 0 to 3.
However, where m, n, or o is an integer of two or more, a plurality of R₁, a plurality of R₂, or a plurality of R₃, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

3. The compound having an azaindeno phenanthrene ring structure according to claim 1,
wherein
the general formula (A-1) is represented by the following general formula (A-3). 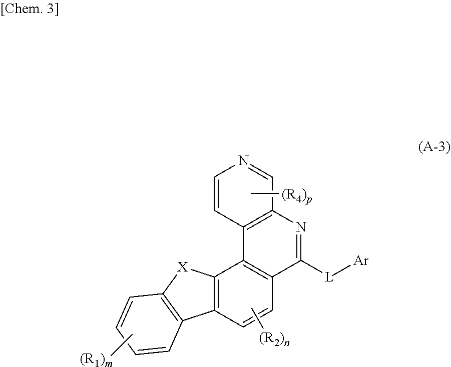
(wherein, L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁, R₂, and R₄ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,
X represents an oxygen atom or a sulfur atom,
m represents an integer of 0 to 4,
n represents an integer of 0 to 2, and
p represents an integer of 0 to 3.
However, where m, n, or p is an integer of two or more, a plurality of R₁, a plurality of R₂, or a plurality of R₄, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

4. The compound having an azaindeno phenanthrene ring structure according to claim 1,
wherein
the general formula (A-1) is represented by the following general formula (A-4). 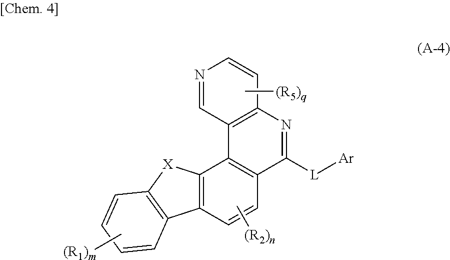
(wherein, L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁, R₂, and R₅ may be the same as or different from each other, and each represents a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,
X represents an oxygen atom or a sulfur atom,
m represents an integer of 0 to 4,
n represents an integer of 0 to 2, and
q represents an integer of 0 to 3.
However, where m, n, or q is an integer of 2 or more, a plurality of R₁, a plurality of R₂, or a plurality of R₅, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

5. The compound having an azaindeno phenanthrene ring structure according to claim 1,
wherein
the general formula (A-1) is represented by the following general formula (A-5). 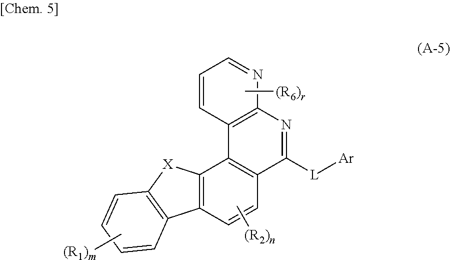
(wherein, L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁, R₂, and R₆ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent,
X represents an oxygen atom or a sulfur atom,
m represents an integer of 0 to 4,
n represents an integer of 0 to 2, and
r represents an integer of 0 to 3.
However, m, n, or r is an integer of two or more, a plurality of R₁, a plurality of R₂, or a plurality of R₆, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

6. The compound having an azaindeno phenanthrene ring structure according to claim 1,
wherein
the general formula (A-1) is represented by the following general formula (A-6). 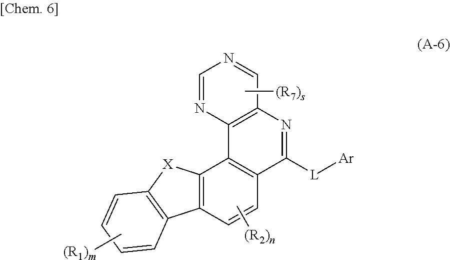
(wherein, L represents a divalent group of a substituted or unsubstituted aromatic hydrocarbon group, a substituted or unsubstituted aromatic heterocyclic group, or a substituted or unsubstituted fused polycyclic aromatic group,
Ar represents a hydrogen atom, a substituted or unsubstituted aromatic hydrocarbon group, a substituted or unsubstituted aromatic heterocyclic group, or a substituted or unsubstituted fused polycyclic aromatic group,
R₁, R₂, and R₇ may be the same as or different from each other, and each represent a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a sub stituent,
X represents an oxygen atom or a sulfur atom,
m represents an integer of 0 to 4,
n represents an integer of 0 to 2, and
s represents an integer of 0 to 2.
However, where m, n, or s is an integer of two or more, a plurality of R₁, a plurality of R₂, or a plurality of R₇, bonded to the same benzene ring, may be the same as or different from each other, and may be bonded with each other via a single bond, a substituted or unsubstituted methylene group, a substituted or unsubstituted amino group, an oxygen atom, or a sulfur atom to form a ring, to the same substituted benzene ring.)

7. The compound having an azaindeno phenanthrene ring structure according to claim 2,
wherein
L in the general formulae (A-2) to (A-6) is represented by the following structural formula (B-1), (B-2), or (B-3), and
two portions among R₁₁ to R₁₅, among R₁₆ to R₂₁, or among R₂₂ to R₂₉ are binding sites. 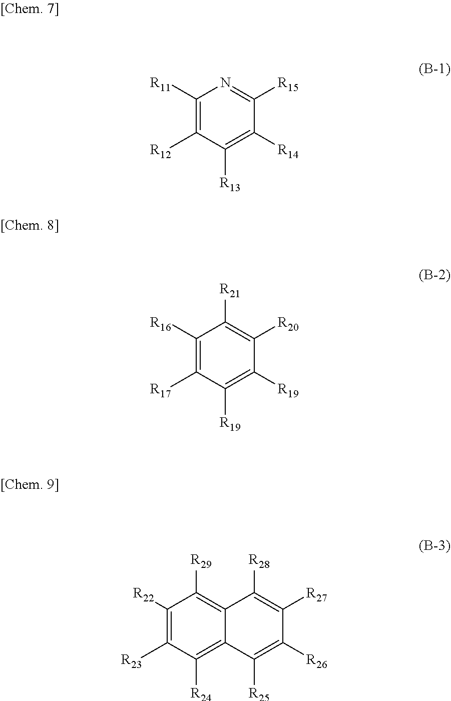
(wherein, R₁₁ to R₂₉ may be the same as or different from each other, and each represent a linking group as a binding site, a hydrogen atom, a deuterium atom, a fluorine atom, a chlorine atom, a cyano group, a nitro group, a trimethylsilyl group, a triphenylsilyl group, a substituted or unsubstituted aromatic hydrocarbon group, a substituted or unsubstituted aromatic heterocyclic group, a substituted or unsubstituted fused polycyclic aromatic group, a linear or branched alkyl group having 1 to 6 carbon atoms which may have a substituent, a cycloalkyl group having 5 to 10 carbon atoms which may have a substituent, a linear or branched alkenyl group having 2 to 6 carbon atoms which may have a substituent, a linear or branched alkyloxy group having 1 to 6 carbon atoms which may have a substituent, or a cycloalkyloxy group having 5 to 10 carbon atoms which may have a substituent.)

8. An organic electroluminescent device, comprising:
a pair of electrodes; and
at least one organic layer sandwiched between the pair of electrodes,
the compound having an azaindeno phenanthrene ring structure according to claim 1 being used as a constituent material of the at least one organic layer.

9. The organic electroluminescent device according to claim 8, wherein
the organic layer containing the compound having an azaindeno phenanthrene ring structure as the constituent material is an electron transport layer.

10. The organic electroluminescent device according to claim 8, wherein
the organic layer containing the compound having an azaindeno phenanthrene ring structure as the constituent material is a hole blocking layer.

11. The organic electroluminescent device according to claim 8, wherein
the organic layer containing the compound having an azaindeno phenanthrene ring structure as the constituent material is a light-emitting layer.

12. The organic electroluminescent device according to claim 8, wherein
the organic layer containing the compound having an azaindeno phenanthrene ring structure as the constituent material is an electron injection layer.
